(19) United States
(12) Patent Application Publication
(10) Pub. No.: US 2009/0124607 A1
(43) Pub. Date: May 14, 2009

(54) Novel Bicyclic Sulfonamides for Use as Glucocorticoid Receptor Modulators in the Treatment of Inflammatory Diseases

(76) Inventors: Hakan Bladh, Lund (SE); Jan Dahmen, Lund (SE); Thomas Hansson, Lund (SE); Krister Henriksson, Lund (SE); Matti Lepisto, Lund (SE); Stinabritt Nilsson, Lund (SE)

Correspondence Address: FISH & RICHARDSON P.C., P.O BOX 1022, MINNEAPOLIS, MN, 55440-1022, US

(21) Appl. No.: 12/090,442

(22) PCT Filed: Oct. 18, 2006
(86) PCT No.: PCT/SE2006/001181

§ 371 Date: Aug. 14, 2008

(30) Foreign Application Priority Data
Oct. 20, 2005 (SE) 0502325-4
Apr. 3, 2006 (SE) 0600747-0

(51) Int. Cl. A61K 31/55; C07D 231/56; A61K 31/416; C07D 401/02; A61K 31/4439; A61K 31/437; A61P 29/00; C07D 239/24; A61K 31/506; C07D 487/02; A61K 31/519

(52) U.S. Cl. 514/220; 548/361.1; 514/403; 546/275.7; 514/338; 544/322; 514/256; 544/262; 514/262.1; 514/304

(57) ABSTRACT

Compounds of formula (I): or a pharmaceutically acceptable salt thereof; compositions comprising them, processes for preparing them and their use in medical therapy (for example modulating the glucocorticoid receptor in a warm blooded animal).

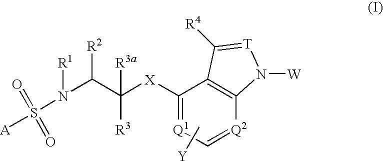

The present invention relates to the use of sulphonamide derivatives as medicaments (for example in the treatment of an inflammatory disease state), to pharmaceutical compositions comprising such derivatives, to certain novel derivatives and to processes for preparing such novel derivatives.

Sulphonamide derivatives are disclosed as anti-inflammatories in WO 2004/019935 and WO 2004/050631. Pharmaceutically active sulphonamides are also disclosed in Arch. Pharm. (1980) 313 166-173, J. Med. Chem. (2003) 46 64-73, J. Med. Chem. (1997) 40 996-1004, EP 0031954, EP 1190710 (WO 200124786), U.S. Pat. No. 5,861,401, U.S. Pat. No. 4,948,809, U.S. Pat. No. 3,992,441 and WO 99/33786.

It is known that certain non-steroidal compounds interact with the glucocorticoid receptor (GR) and, as a result of this interaction, produce a suppression of inflammation (see, for example, U.S. Pat. No. 6,323,199). Such compounds can show a clear dissociation between anti-inflammatory and metabolic actions making them superior to earlier reported steroidal and non-steroidal glucocorticoids. The present invention provides further non-steroidal compounds as modulators (for example agonists, antagonists, partial agonists or partial antagonists) of the glucocorticoid receptor capable of having a dissociation between their anti-inflammatory and metabolic actions.

The present invention provides a compound of formula (I):

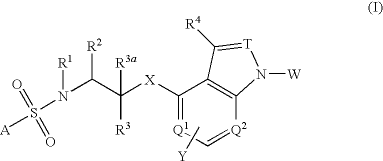

wherein:
A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ haloalkyl, C₁₋₄ haloalkoxy, C₃₋₆ cycloalkyl, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹), pyrazolyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹) or tetrahydrofuranyl (optionally substituted by C₁₋₆ alkyl);
R¹⁰, R¹¹, R¹⁴, R¹⁵, R¹⁶, R¹⁷, R¹⁸, R¹⁹, R²⁰ and R²¹ are, independently, hydrogen, C₁₋₄ alkyl or C₃₋₇ cycloalkyl;
R¹ is hydrogen;
R² is hydrogen, C₁₋₄ alkyl or C₁₋₄ haloalkyl, C₃₋₇ cycloalkyl or C₃₋₇ cyclohaloalkyl;
R³ is hydrogen, C₁₋₄ alkyl or C₁₋₄ haloalkyl;
R^{3a} is hydrogen or C₁₋₄ alkyl;
R⁴ is hydrogen, halogen, C₁₋₄ alkyl or C₁₋₄ haloalkyl;

### T is CH or N;

### Q¹ is CY¹ or N;

### Q² is CY² or N;

W is phenyl, C₃₋₇ cycloalkyl, thienyl, isoxazolyl, pyrazolyl, pyridinyl or pyrimidinyl all of which are optionally substituted by halo, C₁₋₆ alkyl (optionally substituted by C₁₋₆ alkoxy), C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³;

### X is CH₂, O, S, S(O), S(O)₂ or NH;

Y, Y¹ and Y² are, independently, hydrogen, halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR²²R²³;
R¹², R¹³, R²² and R²³ are, independently, hydrogen, C₁₋₄ alkyl or C₃₋₇ cycloalkyl;
or a pharmaceutically acceptable salt thereof.

Compounds of formula (I) can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, trifluoroacetate, fumarate, maleate, tartrate, citrate, oxalate, methanesulphonate, p-toluenesulphonate, succinate, glutarate or malonate.

The compounds of formula (I) may exist as solvates (such as hydrates) and the present invention covers all such solvates.

Alkyl groups and moieties are straight or branched chain and are, for example, methyl, ethyl, n-propyl, iso-propyl, n-butyl, sec-butyl or tert-butyl.

Haloalkyl comprises, for example, 1 to 6, such as 1, 2, 3, 4 or 5 halogen (such as fluorine or chlorine) atoms. It is, for example, CHF₂, CF₃, CH₂CF₃, C₂F₅ or CH₂Cl. Haloalkoxy comprises, for example, 1 to 6, such as 1, 2, 3, 4 or 5 halogen (such as fluorine or chlorine) atoms. It is, for example, OCHF₂, OCF₃, OCH₂CF₃, OC₂F₅ or OCH₂Cl.

Fluoroalkyl comprises, for example, 1 to 6, such as 1, 2, 3, 4 or 5 fluorine atoms. It is, for example, CHF₂, CF₃, CH₂CF₃ or C₂F₅. Fluoroalkoxy comprises, for example, 1 to 6, such as 1, 2, 3, 4 or 5 fluorine atoms. It is, for example, OCHF₂, OCF₃, OCH₂CF₃ or OC₂F₅.

Cycloalkyl is for example, cyclopropyl, cyclopentyl or cyclohexyl.

In one particular aspect the present invention provides a compound of formula (I) wherein: A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ haloalkyl, C₁₋₄ haloalkoxy, C₃₋₆ cycloalkyl, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹) or pyrazolyl (optionally substituted by halo, C₁₋₆ alkyl, C₁₋₆ alkoxy, C₁₋₄ alkylthio, C₁₋₄ is haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹); R¹⁰, R¹¹, R¹⁴, R⁵, R¹⁶, R¹⁷, R¹⁸, R¹⁹, R²⁰ and R²¹ are, independently, hydrogen, C₁₋₄ alkyl or C₃₋₇ cycloalkyl; R¹ is hydrogen; R² is hydrogen, C₁₋₄ alkyl or C₁₋₄ haloalkyl, C₃₋₇ cycloalkyl or C₃₋₇ cyclohaloalkyl; R³ is hydrogen, C₁₋₄ alkyl or C₁₋₄ haloalkyl; R^{3a} is hydrogen; R⁴ is hydrogen, halogen, C₁₋₄ alkyl or C₁₋₄ haloalkyl; T is CH or N; Q¹ is CY¹ or N; Q² is CY² or N; W is phenyl, C₃₋₇ cycloalkyl, thienyl, isoxazolyl, pyrazolyl, pyridinyl or pyrimidinyl all of which are optionally substituted by halo, C₁₋₆ alkyl (optionally substituted by C₁₋₆ alkoxy), C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; X is CH₂, O, S, S(O), S(O)₂ or NH; Y, Y¹ and Y² are, independently, hydrogen, halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR²²R²³; R¹², R¹³, R²² and R²³ are, independently, hydrogen, C₁₋₄ alkyl or C₃₋₇ cycloalkyl; or a pharmaceutically acceptable salt thereof.

In another aspect the present invention provides a compound of formula (I) wherein: A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ fluoroalkyl, C₁₋₄ fluoroalkoxy, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹) or pyrazolyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹); R¹⁰, R¹¹, R¹⁴, R¹⁵, R¹⁶, R¹⁷, R¹⁸, R⁹, R²⁰ and R²¹ are, independently, hydrogen, C₁₋₄ alkyl or C₃₋₇ cycloalkyl; R¹ is hydrogen, C₁₋₆ alkyl, phenyl, pyridinylC(O), C₃₋₆ cycloalkyl, (C₃₋₆ cycloalkyl)CH₂ or C₃₋₄ alkenyl; R² is C₁₋₄ alkyl or C₁₋₄ haloalkyl; R³, R^{3a} and R⁴ are all hydrogen; T is CH or N; Q¹ is CY¹; Q² is CY²; W is phenyl or pyridinyl either of which is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; X is CH₂, O, S, S(O), S(O)₂ or NH; Y, Y¹ and Y² are, independently, hydrogen, halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR²²R²³; provided that two of Y, Y¹ and Y² are hydrogen; R¹², R¹³, R²¹ and R²³ are, independently, hydrogen, C₁₋₄ alkyl or C₃₋₇ cycloalkyl; or a pharmaceutically acceptable salt thereof.

In yet another aspect the present invention provides a compound of formula (I) wherein A is pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ haloalkyl, C₁₋₄ haloalkoxy, C₃₋₆ cycloalkyl, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹), pyrazolyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹) or tetrahydrofuranyl (optionally substituted by C₁₋₆ alkyl).

In a further aspect the present invention provides a compound of formula (I) wherein A is phenyl, and A is optionally substituted by halo, C₁₋₆ alkyl, C₁₋₆ alkoxy, C₁₋₄ alkylthio, C₁₋₄ haloalkyl, C₁₋₄ haloalkoxy, C₃₋₆ cycloalkyl, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, is C(O)NH₂, C(O)NH(C₁₋₄ alkyl), C(O)N(C₁₋₄ alkyl)₂, NHC(O)(C₁₋₄ alkyl) or NR¹⁸R¹⁹), pyrazolyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹) or tetrahydrofuranyl (optionally substituted by C₁₋₆ alkyl).

In another aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl, C₃₋₆ cycloalkyl or phenyl (itself optionally substituted by halogen, C₁₋₄ alkyl, C₁₋₄ haloalkyl, C₁₋₄ alkoxy or C₁₋₄ haloalkoxy)).

In yet another aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy).

In a further aspect the present invention provides a compound of formula (I) wherein pyridyl (optionally substituted by halogen, C₁₋₄ alkyl, C₁₋₄ haloalkyl, C₁₋₄ alkoxy or C₁₋₄ haloalkoxy).

In a still further aspect the present invention provides a compound of formula (I) wherein A is pyrazolyl (optionally substituted by C₁₋₄ alkyl, C₁₋₄ haloalkyl, C₃₋₆ cycloalkyl or phenyl (itself optionally substituted by halogen, C₁₋₄ alkyl, C₁₋₄ haloalkyl, C₁₋₄ alkoxy or C₁₋₄ haloalkoxy)).

In another aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)).

In another aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy).

In a further aspect the present invention provides a compound of formula (I) is wherein A is phenyl (optionally substituted by C₁₋₄ alkyl) or pyrazolyl (optionally substituted by C₁₋₄ alkyl or C₃₋₆ cycloalkyl).

In a still further aspect the present invention provides a compound of formula (I) wherein A is phenyl (optionally substituted by C₁₋₄ alkyl).

In another aspect the present invention provides a compound of formula (I) wherein A is pyrazolyl (optionally substituted by C₁₋₄ alkyl or C₃₋₆ cycloalkyl).

In yet another aspect the present invention provides a compound of formula (I) wherein R¹ is hydrogen.

In a further aspect the present invention provides a compound of formula (I) wherein R² is methyl, ethyl, or C₁₋₂ fluoroalkyl (such as CF₃). In another aspect R² is methyl.

In a still further aspect the present invention provides a compound of formula (I) wherein R³ is hydrogen or C₁₋₄ alkyl (for example methyl). In another aspect R³ is hydrogen.

In another aspect the present invention provides a compound of formula (I) wherein R³¹ is hydrogen.

In a further aspect the present invention provides a compound of formula (I) wherein R⁴ is hydrogen.

In a still further aspect the present invention provides a compound of formula (I) wherein T is N.

In another aspect the present invention provides a compound of formula (I) wherein Y is hydrogen, halogen, C₁₋₄ alkyl, C₁₋₄ haloalkyl, C₁₋₄ alkoxy or C₁₋₄ haloalkoxy. In a further aspect Y is hydrogen.

In yet another aspect the present invention provides a compound of formula (I) wherein Q¹ is CY¹ or N (for example Q¹ is CY¹), wherein Y¹ is hydrogen, halogen or C₁₋₄ alkyl. In another aspect Y¹ is hydrogen.

In a further aspect the present invention provides a compound of formula (I) wherein Q² is CY² or N (for example Q² is CY²), wherein Y² is hydrogen or halogen. In another aspect Y² is hydrogen.

In a still further aspect the present invention provides a compound of formula (I) wherein Q¹ and Q² are both CH; T is N; and Y and R⁴ are both hydrogen.

In another aspect the present invention provides a compound of formula (I) wherein W is phenyl optionally substituted by halo, C₁₋₆ alkyl (optionally substituted by C₁₋₆ alkoxy), C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; and R¹² and R¹³ are, independently, hydrogen, C₁₋₄ alkyl or C₃₋₇ cycloalkyl.

In yet another aspect the present invention provides a compound of formula (I) wherein W is thienyl, isoxazolyl, pyrazolyl, pyridinyl or pyrimidinyl all of which are optionally substituted by halo, C₁₋₆ alkyl (optionally substituted by C₁₋₆ alkoxy), C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; and R¹² and R¹³ are, independently, hydrogen, C₁₋₄ alkyl or C₃₋₇ cycloalkyl.

In another aspect the present invention provides a compound of formula (I) wherein W is phenyl, pyridinyl or pyrimidinyl all of which are optionally substituted by halogen, C₁₋₄ alkyl (optionally substituted by C₁₋₄ alkoxy), C₁₋₄ alkoxy, C₁₋₄ fluoroalkyl, C₁₋₄ fluoroalkoxy, CN or CO₂H.

In yet another aspect the present invention provides a compound of formula (I) wherein W is pyridinyl or pyrimidinyl either of which is optionally substituted by halogen, C₁₋₄ alkyl (optionally substituted by C₁₋₄ alkoxy), C₁₋₄ alkoxy, C₁₋₄ fluoroalkyl, C₁₋₄ fluoroalkoxy, CN or CO₂H.

In a further aspect the present invention provides a compound of formula (I) wherein W is phenyl optionally substituted by halogen, C₁₋₄ alkyl (optionally substituted by C₁₋₄ alkoxy), C₁₋₄ alkoxy, C₁₋₄ fluoroalkyl, C₁₋₄ fluoroalkoxy, CN or CO₂H.

In a still further aspect the present invention provides a compound of formula (I) wherein W is phenyl or pyridinyl either of which is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

In another aspect the present invention provides a compound of formula (I) wherein W is phenyl optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or is C(O)NH₂.

In yet another aspect the present invention provides a compound of formula (I) wherein W is pyridinyl optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

In a further aspect the present invention provides a compound of formula (I) wherein A is phenyl optionally substituted by C₁₋₄ alkyl (such as methyl); R¹, R³ and R⁴ are all hydrogen; R² is methyl; X is O or NH; Y is hydrogen; T is N; Q¹ and Q² are both CH; and W is phenyl or pyridinyl either of which is optionally substituted by halogen (such as fluoro).

In a still further aspect the present invention provides a compound of formula (I) wherein: A is phenyl (optionally substituted by C₁₋₄ alkyl) or pyrazolyl (optionally substituted by C₁₋₄ alkyl or C₃₋₆ cycloalkyl); R² is hydrogen, C₁₋₄ alkyl or CF₃; R³ is hydrogen or C₁₋₄ alkyl; Q¹ is CY¹ or N; wherein Y¹ is hydrogen, halogen or C₁₋₄ alkyl; Q² is CY² or N; wherein Y² is hydrogen or halogen; T is N; Y, R¹ and R⁴ are both hydrogen; W is phenyl, pyridinyl or pyrimidinyl all of which are optionally substituted by halogen, C₁₋₄ alkyl (optionally substituted by C₁₋₄ alkoxy), C₁₋₄ alkoxy, C₁₋₄ fluoroalkyl, C₁₋₄ fluoroalkoxy, CN or CO₂H; or a pharmaceutically acceptable salt thereof.

In a further aspect the present invention provides each individual compound:
N-((1S)-2-{[1-(4-Fluorophenyl)-1H-indazol-4-yl]oxy}-1-methylethyl)-2,4,6-trimethylbenzenesulfonamide;
N-[(1S)-2-[[1-(4-Fluorophenyl)-1H-indazol-4-yl]amino]-1-methylethyl]-2,4,6-trimethyl-benzenesulfonamide;
N-((1S)-2-{[1-(6-Fluoropyridin-3-yl)-1H-indazol-4-yl]amino}-1-methylethyl)-2,4,6-trimethylbenzenesulfonamide;
2,4,6-Trimethyl-N-[2,2,2-trifluoro-1-({[1-(6-fluorophenyl)-1H-indazol-4-yl]oxy}methyl)ethyl]benzenesulfonamide;
N-((1S)-2-{[1-(4-Methoxyphenyl)-1H-indazol-4-yl]amino}-1-methylethyl)-2,4,6-trimethylbenzenesulfonamide;
2,4,6-Trimethyl-N-[(1S)-1-methyl-2-({1-[3-(trifluoromethoxy)phenyl]-1H-indazol-4-yl}amino)ethyl]benzenesulfonamide;
2,4,6-Trimethyl-N-{(1S)-1-methyl-2-[(1-phenyl-1H-indazol-4-yl)amino]ethyl}benzenesulfonamide;
N-((1S)-2-{[1-(3-Methoxyphenyl)-1H-indazol-4-yl]amino}-1-methylethyl)-2,4,6-trimethylbenzenesulfonamide;
2,4,6-Trimethyl-N-((1S)-1-methyl-2-{[1-(3-methylphenyl)-1H-indazol-4-yl]amino}ethyl)benzenesulfonamide;
N-((1S)-2-{[1-(2-Fluoropyridin-4-yl)-1H-indazol-4-yl]amino}-1-methylethyl)-2,4,6-trimethylbenzenesulfonamide;
N-((1S)-2-{[1-(6-Methoxypyridin-3-yl)-1H-indazol-4-yl]amino}-1-methylethyl)-2,4,6-trimethylbenzenesulfonamide;
2,4,6-Trimethyl-N-((1S)-1-methyl-2-{[1-(4-methylphenyl)-1H-indazol-4-yl]amino}ethyl)benzenesulfonamide;
N-((1S)-2-{[1-(3-Fluorophenyl)-1H-indazol-4-yl]amino}-1-methylethyl)-2,4,6-trimethylbenzenesulfonamide;
2,4,6-Trimethyl-N-{(1S)-1-methyl-2-[(1-pyridin-4-yl-1H-indazol-4-yl)amino]ethyl}benzenesulfonamide;
2,4,6-Trimethyl-N-{(1S)-1-methyl-2-[(1-pyrimidin-5-yl-1H-indazol-4-yl)amino]ethyl}benzenesulfonamide;
2,4,6-Trimethyl-N-{(1S)-1-methyl-2-[(1-pyridin-3-yl-1H-indazol-4-yl)amino]ethyl}benzenesulfonamide;
N-((1S)-2-{[1-(4-Fluoro-3-methylphenyl)-1H-indazol-4-yl]amino}-1-methylethyl)-2,4,6-trimethylbenzenesulfonamide;
3-[4-({(2S)-2-[(2,4,6-Benzenesulfonyl)amino]propyl}amino)-1H-indazol-1-yl]benzoic acid;
2,4,6-Trimethyl-N-[(1S)-1-methyl-2-({1-[3-(trifluoromethyl)phenyl]-1H-indazol-4-yl}amino)ethyl]benzenesulfonamide;
N-[(1S)-2-({1-[3-(Methoxymethyl)phenyl]-1H-indazol-4-yl}amino)-1-methylethyl]-2,4,6-trimethylbenzenesulfonamide;
N-((1S)-2-{[1-(3-Fluoro-4-methoxyphenyl)-1H-indazol-4-yl]amino}-1-methylethyl)-2,4,6-trimethylbenzenesulfonamide;
N-((1S)-2-{[1-(4-Chlorophenyl)-1H-indazol-4-yl]amino}-1-methylethyl)-2,4,6-trimethylbenzenesulfonamide;
N-((1S)-2-{[1-(4-Fluorophenyl)-5-methyl-1H-indazol-4-yl]amino}-1-methylethyl)-2,4,6-trimethylbenzenesulfonamide;
N-((2R)-2-{[1-(4-Fluorophenyl)-1H-indazol-4-yl]amino}propyl)-2,4,6-trimethylbenzenesulfonamide;
1-Cyclopentyl-N-((1S)-2-{[1-(4-fluorophenyl)-1H-indazol-4-yl]amino}-1-methylethyl)-3,5-dimethyl-1H-pyrazole-4-sulfonamide;
1-Cyclopentyl-N-((1S)-2-{[1-(6-fluoropyridin-3-yl)-1H-indazol-4-yl]amino}-1-methylethyl)-3,5-dimethyl-1H-pyrazole-4-sulfonamide;
1-Cyclopentyl-3,5-dimethyl-N-[(1S)-1-methyl-2-({1-[4-(trifluoromethoxy)phenyl]-1H-indazol-4-yl}amino)ethyl]-1H-pyrazole-4-sulfonamide;
1-Cyclopentyl-N-((1S)-2-{[1-(2-methoxypyrimidin-5-yl)-1H-indazol-4-yl]amino}-1-methylethyl)-3,5-dimethyl-1H-pyrazole-4-sulfonamide;
1-Cyclopentyl-3,5-dimethyl-N-{(1S)-1-methyl-2-[(1-pyrimidin-5-yl-1H-indazol-4-yl)amino]ethyl}-1H-pyrazole-4-sulfonamide;
N-((1S)-2-{[1-(4-Cyanophenyl)-1H-indazol-4-yl]amino}-1-methylethyl)-1-cyclopentyl-3,5-dimethyl-1H-pyrazole-4-sulfonamide;
1-Cyclopentyl-N-((1S)-2-{[1-(5-methoxypyridin-3-yl)-1H-indazol-4-yl]amino}-1-methylethyl)-3,5-dimethyl-1H-pyrazole-4-sulfonamide;
N-((1S)-2-{[5-Fluoro-1-(4-fluorophenyl)-1H-indazol-4-yl]amino}-1-methylethyl)-2,4,6-trimethylbenzenesulfonamide;
N-((1S)-2-{[7-Fluoro-1-(4-fluorophenyl)-1H-indazol-4-yl]amino}-1-methylethyl)-2,4,6-trimethylbenzenesulfonamide;
2,4,6-Trimethyl-N-{(1S)-1-methyl-2-[(1-phenyl-1H-pyrazolo[3,4-d]pyrimidin-4-yl)amino]ethyl}benzenesulfonamide;
N-[(1S)-1-({[1-(4-Fluorophenyl)-1H-indazol-4-yl]amino}methyl)-2-methylpropyl]-2,4,6-trimethylbenzenesulfonamide;
N-[2-[1-(4-Fluorophenyl)indazol-4-yl]sulfanyl-1-methylethyl]-2,4,6-trimethyl-benzenesulfonamide;
N-[2-[1-(4-Fluorophenyl)indazol-4-yl]sulfonyl-1-methylethyl]-2,4,6-trimethyl-benzenesulfonamide;
N-{3-[1-(4-Fluorophenyl)-1H-indazol-4-yl]-1-methylpropyl}-2,4,6-trimethylbenzenesulfonamide;
N-{(1S)-3-[1-(4-Fluorophenyl)-1H-indazol-4-yl]-1-methylpropyl}-2,4,6-trimethylbenzenesulfonamide;
N-((2S)-2-{[1-(4-Fluorophenyl)-1H-indazol-4-yl]amino}propyl)-2,4,6-trimethylbenzenesulfonamide;
N-((1S)-2-{[1-(4-Fluorophenyl)-1H-indazol-4-yl]oxy}-1-methylethyl)-3,5-dimethyl-1H-pyrazole-4-sulfonamide;
3,5-Dimethyl-N-{(1S)-1-methyl-2-[(1-pyridin-3-yl-1H-indazol-4-yl)oxy]ethyl}-1H-pyrazole-4-sulfonamide;
1-tert-Butyl-N-((1S)-2-{[1-(4-fluorophenyl)-1H-indazol-4-yl]oxy}-1-methylethyl)-3,5-dimethyl-1H-pyrazole-4-sulfonamide;
1-tert-Butyl-3,5-dimethyl-N-{(1S)-1-methyl-2-[(1-pyridin-3-yl-1H-indazol-4-yl)oxy]ethyl}-1H-pyrazole-4-sulfonamide;
N-((1S)-2-{[1-(4-Fluorophenyl)-1H-indazol-4-yl]oxy}-1-methylethyl)-3,5-dimethyl-1-[(3R)-tetrahydrofuran-3-yl]-1H-pyrazole-4-sulfonamide;
1-(1-Ethylpropyl)-N-((1S)-2-{[1-(4-fluorophenyl)-1H-indazol-4-yl]oxy}-1-methylethyl)-3,5-dimethyl-1H-pyrazole-4-sulfonamide;
N-((1S)-2-{[1-(4-Fluorophenyl)-1H-indazol-4-yl]oxy}-1-methylethyl)-3,5-dimethyl-1-[(3S)-tetrahydrofuran-3-yl]-1H-pyrazole-4-sulfonamide;
1-Cyclopentyl-N-((1S)-2-{[1-(4-fluorophenyl)-1H-indazol-4-yl]oxy}-1-methylethyl)-3,5-dimethyl-1H-pyrazole-4-sulfonamide;
1-Cyclopentyl-3,5-dimethyl-N-{(1S)-1-methyl-2-[(1-pyridin-3-yl-1H-indazol-4-yl)oxy]ethyl}-1H-pyrazole-4-sulfonamide;
N-{(1S)-2-[(1-Cyclopentyl-1H-indazol-4-yl)amino]-1-methylethyl}-2,4,6-trimethylbenzenesulfonamide;
N-((1S)-1-ethyl-2-{[1-(4-methylphenyl)-1H-indazol-4-yl]amino}ethyl)benzenesulfonamide;
N-((1S)-2-{[1-(4-Fluorophenyl)-3-methyl-1H-indazol-4-yl]amino}-1-methylethyl)-2,4,6-trimethylbenzenesulfonamide;
N-{(1S)-3-[3-(4-Fluorophenyl)-1H-indazol-7-yl]-1-methylpropyl}-2,4,6-trimethylbenzenesulfonamide;
2,4,6-Trimethyl-N-[(1S)-1-methyl-3-(1-pyrimidin-5-yl-1H-indazol-4-yl)propyl]benzenesulfonamide; or,
N-[2-[[1-(4-Fluorophenyl)indazol-4-yl]amino]-2-methylpropyl]-2,4,6-trimethyl-benzenesulfonamide;
   or a pharmaceutically acceptable salt thereof.

The compounds of formula (I) can be prepared using or adapting methods disclosed in the art, or by using or adapting the methods disclosed in the Examples below. Starting materials for the preparative methods are either commercially available or can be prepared by using or adapting literature methods.

For example a compound of formula (I) can be prepared by coupling a compound of formula (II):

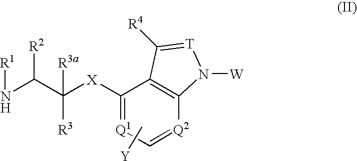

with a compound of formula (III):

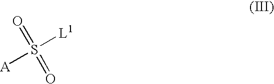

wherein L¹ is a leaving group (such as halogen (for example chloro) or mesylate or tosylate), in a suitable solvent (such as THF or DMF), in the presence of a suitable base (such as a tri(C₁₋₆ alkyl)amine, for example diisopropylethylamine) and at a suitable temperature (such as −10 to 50° C.).

Alternatively, a compound of formula (I) can be prepared by coupling a compound of formula (IV):

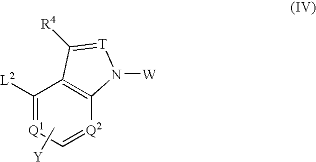

wherein L² is a leaving group (such as halogen, mesylate or tosylate) with a compound of formula (V):

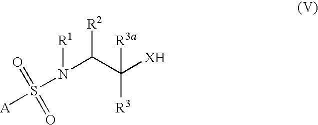

in a suitable solvent (such as an aromatic solvent, for example toluene), in the presence of a suitable base (such as a alkali metal alkoxide (for example sodium tert-butoxide) or, sodium hydride (for example when X is CH₂)) at a suitable temperature (for example in the range 80 to 120° C.).

Alternatively, a compound of formula (I) can be prepared by coupling a compound of formula (VI):

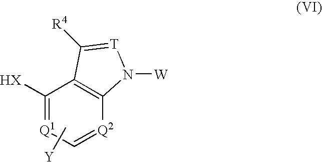

with a compound of formula (III):

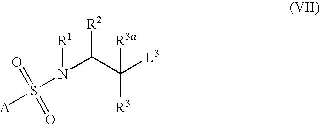

wherein L³ is a leaving group (such as halogen, mesylate or tosylate), in a suitable solvent (such as DMF or acetonitrile), in the presence of a suitable base (such as an alkali metal carbonate, for example cesium carbonate or potassium carbonate) at a suitable temperature (for example in the range 50 to 150° C.).

The invention further provides processes for the preparation of these compounds of formula (I).

Because of their ability to bind to the glucocorticoid receptor the compounds of formula (I) are useful as anti-inflammatory agents, and can also display antiallergic, immunosuppressive and anti-proliferative actions. Thus, a compound of formula (I), or a pharmaceutically acceptable salt thereof can be used as a medicament for the treatment or prophylaxis of one or more of the following pathologic conditions (disease states) in a mammal (such as a human):
(i) Lung diseases, which coincide with inflammatory, allergic and/or proliferative processes:
   chronically obstructive lung diseases of any origin, mainly bronchial asthma
   bronchitis of different origins
   all forms of restructive lung diseases, mainly allergic alveolitis
   all forms of pulmonary edema, mainly toxic pulmonary edema
   sarcoidoses and granulomatoses, such as Boeck's disease
(ii) Rheumatic diseases/auto-immune diseases/degenerative joint diseases, which coincide with inflammatory, allergic and/or proliferative processes:
   all forms of rheumatic diseases, especially rheumatoid arthritis, acute rheumatic fever, polymyalgia rheumatica, collagenoses
   reactive arthritis
   inflammatory soft-tissue diseases of other origins
   arthritic symptoms in degenerative joint diseases (arthroses)
   traumatic arthritides
   collagen diseases of other origins, for example systemic lupus erythematodes, sclerodermia, polymyositis, dermatomyositis, polyarteritis nodosa, temporal arteritis
   Sjögren's syndrome, Still syndrome, Felty's syndrome
(iii) Allergies, which coincide with inflammatory, allergic and/or proliferative processes:
   All forms of allergic reactions, for example Quincke's edema, hay fever, insect bites, allergic reactions to pharmaceutical agents, blood derivatives, contrast media, etc., anaphylactic shock, urticaria, contact dermatitis
(iv) Dermatological diseases, which coincide with inflammatory, allergic and/or proliferative processes:
   atopic dermatitis (mainly in children)
   psoriasis
   erythematous diseases, triggered by different noxae, for example radiation, chemicals, burns, etc.
   acid burns
   bullous dermatoses
   diseases of the lichenoid group
   itching (for example of allergic origins)
   seborrheal eczema
   rosacea
   pemphigus vulgaris
   erythema exudativum multiforme
   erythema nodosum
   balanitis
   vulvitis
   inflammatory hair loss, such as alopecia areata
   cutaneous T-cell lymphoma
(v) Nephropathies, which coincide with inflammatory, allergic and/or proliferative processes:
   nephrotic syndrome
   all nephritides
(vi) Liver diseases, which coincide with inflammatory, allergic and/or proliferative processes:
   acute liver cell decomposition
   acute hepatitis of different origins, for example virally-, toxically- or pharmaceutical agent-induced
   chronically aggressive and/or chronically intermittent hepatitis
(vii) Gastrointestinal diseases, which coincide with inflammatory, allergic and/or proliferative processes:
   regional enteritis (Crohn's disease)
   ulcerative colitis
   gastroenteritis of other origins, for example native sprue
(viii) Proctological diseases, which coincide with inflammatory, allergic and/or proliferative processes:
   anal eczema
   fissures
   haemorrhoids
   idiopathic proctitis
(ix) Eye diseases, which coincide with inflammatory, allergic and/or proliferative processes:
   allergic keratitis, uvenitis iritis
   conjunctivitis
   blepharitis
   optic neuritis
   chorioiditis
   sympathetic ophthalmia
(x) Diseases of the ear-nose-throat area, which coincide with inflammatory, allergic and/or proliferative processes:
   allergic rhinitis, hay fever
   otitis externa, for example caused by contact dermatitis, infection, etc.
   otitis media
(xi) Neurological diseases, which coincide with inflammatory, allergic and/or proliferative processes:
   cerebral edema, mainly tumor-induced cerebral edema
   multiple sclerosis
   acute encephalomyelitis
   different forms of convulsions, for example infantile nodding spasms
(xii) Blood diseases, which coincide with inflammatory, allergic and/or proliferative processes:
   acquired haemolytic anemia
   idiopathic thrombocytopenia
(xiii) Tumor diseases, which coincide with inflammatory, allergic and/or proliferative processes:
   acute lymphatic leukaemia
   malignant lymphoma
   lymphogranulomatoses
   lymphosarcoma
   extensive metastases, mainly in breast and prostate cancers
(xiv) Endocrine diseases, which coincide with inflammatory, allergic and/or proliferative processes:
   endocrine orbitopathy
   thyrotoxic crisis
   de Quervain's thyroiditis
   Hashimoto's thyroiditis
   hyperthyroidism
(xv) Transplants, which coincide with inflammatory, allergic and/or proliferative processes;
(xvi) Severe shock conditions, which coincide with inflammatory, allergic and/or proliferative processes, for example anaphylactic shock
(xvii) Substitution therapy, which coincides with inflammatory, allergic and/or proliferative processes, with:
   innate primary suprarenal insufficiency, for example congenital adrenogenital syndrome
   acquired primary suprarenal insufficiency, for example Addison's disease, autoimmune adrenalitis, meta-infective, tumors, metastases, etc.
   innate secondary suprarenal insufficiency, for example congenital hypopituitarism
   acquired secondary suprarenal insufficiency, for example meta-infective, tumors, etc.
(xviii) Emesis, which coincides with inflammatory, allergic and/or proliferative processes:
   for example in combination with a 5-HT₃-antagonist in cytostatic-agent-induced vomiting.

Without prejudice to the foregoing, the compounds of formula (I) can also be used to treat disorders such as: Conies Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, oesophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cinhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis Sweet's disease, type 1 reactive leprosy, capillary hemangiomas, lichen planus, erythema nodosum acne, hirsutism, toxic epidermal necrolysis, erythema multiform, cutaneous T-cell lymphoma, psychoses, cognitive disorders (such as memory disturbances) mood disorders (such as depression and bipolar disorder), anxiety disorders and personality disorders.

As used herein the term “congestive heart failure” (CHF) or “congestive heart disease” refers to a disease state of the cardiovascular system whereby the heart is unable to efficiently pump an adequate volume of blood to meet the requirements of the body's tissues and organ systems. Typically, CHF is characterized by left ventricular failure (systolic dysfunction) and fluid accumulation in the lungs, with the underlying cause being attributed to one or more heart or cardiovascular disease states including coronary artery disease, myocardial infarction, hypertension, diabetes, valvular heart disease, and cardiomyopathy. The term “diastolic congestive heart failure” refers to a state of CHF characterized by impairment in the ability of the heart to properly relax and fill with blood. Conversely, the term “systolic congestive heart failure” refers to a state of CHF characterized by impairment in the ability of the heart to properly contract and eject blood.

As will be appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of physiological disorders contemplates both acute events and chronic conditions. In an acute event, compound is administered at the onset of symptoms and discontinued when the symptoms disappear.

In another aspect the present invention provides the use of a compound or formula (I), or a pharmaceutically acceptable salt thereof, for use in therapy (such as a therapy described above).

In yet another aspect the present invention provides the use of a compound or formula (I), or a pharmaceutically acceptable salt thereof, in the manufacture of a medicament for use in the treatment of a glucocorticoid receptor mediated disease state (such as a disease state described above).

In a further aspect the invention provides the use of a compound of formula (I), or a pharmaceutically acceptable salt thereof, in the manufacture of a medicament for use in the treatment of an inflammatory (such as an arthritic) condition.

In a still further aspect the invention provides the use of a compound of formula (I), or a pharmaceutically acceptable salt thereof, in the manufacture of a medicament for use in the treatment of an asthmatic condition.

In another aspect the invention provides the use of a compound of formula (I), or a pharmaceutically acceptable salt thereof, in the manufacture of a medicament for use in the treatment of COPD.

The present invention further provides a method of treating a glucocorticoid receptor mediated disease state in a mammal (such as man), which comprises administering to a mammal in need of such treatment an effective amount of a compound of formula (I), or a pharmaceutically acceptable salt thereof.

In order to use a compound of formula (I), or a pharmaceutically acceptable salt thereof, for the therapeutic treatment of a mammal, said active ingredient is normally formulated in accordance with standard pharmaceutical practice as a pharmaceutical composition.

Therefore in another aspect the present invention provides a pharmaceutical composition comprising a compound of formula (I), or a pharmaceutically acceptable salt thereof, (active ingredient) and a pharmaceutically acceptable adjuvant, diluent or carrier. In a further aspect the present invention provides a process for the preparation of said composition comprising mixing the active ingredient with a pharmaceutically acceptable adjuvant, diluent or carrier. Depending on the mode of administration, the pharmaceutical composition can comprise from 0.05 to 99% w (percent by weight), for example from 0.05 to 80% w, such as from 0.10 to 70% w (for example from 0.10 to 50% w), of active ingredient, all percentages by weight being based on total composition.

A pharmaceutical composition of the present invention can be administered in a standard manner for the disease condition that it is desired to treat, for example by topical (such as to the lung and/or airways or to the skin), oral, rectal or parenteral administration. Thus, a the compound of formula (I), or a pharmaceutically acceptable salt thereof, may be formulated into the form of, for example, an aerosol, a powder (for example dry or dispersible), a tablet, a capsule, a syrup, a granule, an aqueous or oily solution or suspension, an (lipid) emulsion, a suppository, an ointment, a cream, drops, or a sterile injectable aqueous or oily solution or suspension.

A suitable pharmaceutical composition of this invention is one suitable for oral administration in unit dosage form, for example a tablet or capsule containing between 0.1 mg and 1 g of active ingredient.

In another aspect a pharmaceutical composition of the invention is one suitable for intravenous, subcutaneous, intraarticular or intramuscular injection.

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. Tablets may be enteric coated by conventional means, for example to provide a coating of cellulose acetate phthalate.

The invention further relates to combination therapies or compositions wherein a GR agonist of formula (I), or a pharmaceutically acceptable salt thereof, or a pharmaceutical composition comprising a GR agonist of formula (I), or a pharmaceutically acceptable salt thereof, is administered concurrently (possibly in the same composition) or sequentially with one or more agents for the treatment of any of the above disease states.

For example, for the treatment of rheumatoid arthritis, osteoarthritis, COPD, asthma or allergic rhinitis a GR agonist of the invention can be combined with one or more agents for the treatment of such a condition. Where such a combination is to be administered by inhalation, then the one or more agents is selected from the list comprising:
a PDE4 inhibitor including an inhibitor of the isoform PDE4D;
a selective β.sub2. adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
a muscarinic receptor antagonist (for example a M1, M2 or M3 antagonist, such as a selective M3 antagonist) such as ipratropium bromide, tiotropium bromide, oxitropium bromide, pirenzepine or telenzepine;
a modulator of chemokine receptor function (such as a CCR1 receptor antagonist); or,
an inhibitor of p38 kinase function.

In another aspect of the invention where such a combination is for the treatment of COPD, asthma or allergic rhinitis the GR agonist of formula (I), or a pharmaceutically acceptable salt thereof, can be administered by inhalation or by the oral route and this is in combination with a xanthine (such as aminophylline or theophylline) which can be administered by inhalation or by the oral route.

The following Examples illustrate the invention. The following abbreviations are used in the Examples:
THF tetrahydrofuran
TFA trifluoroacetic acid
DMSO dimethylsulfoxide
DMF N,N-dimethylformamide
TBAT N,N,N-tributylbutan-1-aminium difluoro(triphenyl)silicate
DIEA diisopropylethyl amine
NMP 1-Methyl-2-pyrrolidinone
BINAP (R)-(+)-2,2′-Bis(diphenylphosphino)-1,1′-binaphthyl
Pd2(dba)₃ Tris(dibenzylideneacetone)dipalladium(0)
LDA lithium diisopropylamide
Pd-18 1,1-bis(di-tery-butylphosphino)ferrocene palladium dichloride

### General Methods

NMR spectra were recorded on a Varian Mercury-VX 300 MHz instrument or a Varian Inova 400 MHz instrument. The central peaks of chloroform-d (H 7.27 ppm), acetone (H 2.05 ppm), dichloromethane-d2 (H 5.32 ppm) or DMSO-d₆ (H 2.50 ppm) were used as internal references.

The following method was used for LC/MS analysis:

Instrument Agilent 1100; Column Waters Symmetry 2.1×30 mm; Mass APCI; Flow rate 0.7 mL/min; Wavelength 254 nm; Solvent A: water+0.1% TFA; Solvent B: acetonitrile+0.1% TFA; Gradient 15-95%/B 2.7 min, 95% B 0.3 min.

The following method was used for GC-MS analysis:

Low resolution mass spectra and accurate mass determination were recorded on a Hewlett-Packard GC. MS system equipped with EI ionisation chamber, 70 eV.

The following method was used for LC analysis:

Method A. Instrument Agilent 1100; Column: Kromasil C18 100×3 mm, 5μ particle size, Solvent A: 0.1% TFA/water, Solvent B: 0.08% TFA/acetonitrile Flow: 1 mL/min, Gradient 10-100%/B 20 min, 100% B 1 min. Absorption was measured at 220, 254 and 280 nm.

A Kromasil KR-100-5-C18 column (250×20 mm, Akzo Nobel) and mixtures of acetonitrile/water (0.1% TFA) at a flow rate of 10 mL/min was used for preparative HPLC. Unless stated otherwise, starting materials were commercially available. All solvents and commercial reagents were of laboratory grade and were used as received.

### EXAMPLE 1

### N-((1S)-2-{[1-(4-Fluoro-phenyl)-1H-indazol-4-yl]oxy}-1-methylethyl)-2,4,6-trimethylbenzenesulfonamide

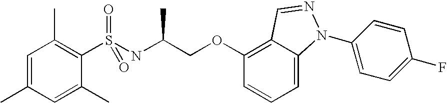

### (2S)-2-[(2,4,6-Benzenesulfonyl)amino]propyl 2,4,6-trimethylbenzenesulfonate

L-Alaninol (4.8 g, 64 mmol) and 2,4,6-benzenesulfonyl chloride (30 g, 137 mmol) were dissolved in 200 mL pyridine and stirred at room temperature overnight. The mixture was evaporated, dissolved in ethyl acetate (200 mL) and washed with 1M HCl, saturated aqueous NaHCO₃. The organic layer was dried, concentrated and purified by silica gel column chromatography (heptane-ethyl acetate).

APCI-MS m/z: 440.1 [MH+].

### 1-(4-Fluorophenyl)-4-methoxy-1H-indazole

2-Fluoro-6-methoxy-benzaldehyde (1 mmol, 154 mg), 4-fluorophenylhydrazine hydrochloride (1 mmol, 162 mg) and sodium tert-butoxide (3 mmol, 336 mg) was diluted in 4 mL NMP and heated to 100° C. for 1 hour. After cooling to room temperature the reaction mixture was diluted with dichloromethane (50 mL) and washed with 1M HCl, saturated aqueous NaHCO₃. The organic phase was dried over Na₂SO₄, concentrated and purified by silica gel column chromatography (heptane-ethyl acetate).

APCI-MS m/z: 243.1 [MH⁺].

### 1-(4-Fluorophenyl)-1H-indazol-4-ol

1-(4-Fluorophenyl)-4-methoxy-1H-indazole (0.5 mmol, 120 mg) was dissolved in dichloromethane (2 mL) and BBr₃ (2 mL, 1M in dichloromethane) was added. The reaction mixture was stirred in room temperature overnight before it was quenched with water (20 mL). The product was extracted with dichloromethane (2×20 mL) and washed with saturated aqueous NaHCO₃. The organic phase was dried over Na₂SO₄, concentrated and purified by silica gel column chromatography (heptane-ethyl acetate).

¹H NMR (400 MHz, DMSO-d₆) δ 10.39 (1H, s), 8.33 (1H, dd,), 7.76 (2H, tt), 7.41 (2H, dd), 7.27 (1H, t), 7.18 (1H, d), 6.56 (1H, d); APCI-MS m/z: 229.1 [MH+].

### N-((1S)-2-{[1-(4-Fluorophenyl)-1H-indazol-4-yl]oxy}-1-methylethyl)-2,4,6-trimethylbenzenesulfonamide

(2S)-2-[(2,4,6-Benzenesulfonyl)amino]propyl 2,4,6-trimethylbenzenesulfonate (167 mg, 0.38 mmol) was added to a slurry containing Cs₂CO₃ (168 mg, 0.5 mmol) and 1-(4-Fluorophenyl)-1H-indazol-4-ol (80 mg, 0.35 mmol) in DMF (4 mL). The reaction mixture was stirred overnight in room temperature before it was diluted with ethyl acetate (20 mL) and washed with 1M HCl. The organic layer was dried, concentrated and purified by HPLC-C₁₈.

¹H NMR (400 MHz, DMSO-d₆) δ 8.07 (1H, s), 7.84-7.72 (3H, m), 7.42 (2H, t), 7.30 (2H, dd), 6.91 (2H, s), 6.50 (1H, dd), 4.01 (1H, dd), 3.89 (1H, dd), 3.63-3.54 (1H, m), 2.55 (6H, s), 2.17 (3H, s), 1.17 (3H, d); APCI-MS m/z: 468.1 [MH+].

### EXAMPLE 2

### N-[(1S)-2-[[1-(4-Fluorophenyl)-1H-indazol-4-yl]amino]-1-methylethyl]-2,4,6-trimethyl-benzenesulfonamide

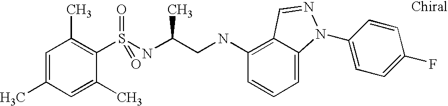

### 3-Bromo-2-methylbenzenediazonium tetrafluoroborate

3-Bromo-2-methylaniline (10 mmol, 1.86 g) was suspended in H₂O (3 mL) and mixed with HCl (37% in H₂O, 25 mL) and stirred for 1 hour in room temperature. The reaction mixture was cooled to −5° C. and NaNO₂ (10 mmol, 672 mg) dissolved in water (3 mL) was added dropwise over a period of 25 minutes followed by a rapid addition of HBF₄ (50%, 18 mL). The temperature was raised to room temperature and the diazonium salt was collected by filtration and washed with dichloromethane. The salt was used in the next step without any further purification.

### 4-Bromo-1H-indazole

3-Bromo-2-methylbenzenediazonium tetrafluoroborate (991 mg, 2.8 mmol) was added in one portion to a stirred mixture of potassium acetate (560 mg, 0.57 mmol) and 18-crown-6 (0.14 mmol, 40 mg) in dichloromethane (25 mL, 4 Å). After stirring at room temperature for one hour the reaction mixture was diluted with dichloromethane (50 mL) and washed with water. The organic layer was dried, concentrated and purified by silica gel column chromatography (heptane-ethyl acetate).

¹H NMR (400 MHz, CDCl₃) δ 9.03 (1H, s), 8.17 (1H, s), 7.52 (1H, d), 7.37 (1H, d), 7.32-7.26 (1H, m); APCI-MS m/z: 197.0, 199.0 [MH⁺].

### 4-Bromo-1-(4-fluorophenyl)-1H-indazole

4-Bromo-1H-indazole (200 mg, 1 mmol) was dissolved in dichloromethane (10 mL, 4 Å) together with (4-fluorophenyl)boronic acid (2 mmol, 278 mg), anhydrous cupric acetate (1 mmol, 180 mg) and pyridine (2 mmol, 190 μL). The reaction mixture was stirred overnight and directly purified by silica gel column chromatography (heptane-ethyl acetate).

APCI-MS m/z: 290.9, 292.9 [MH+].

### (2S)-2-[(2,4,6-Trimethylbenzenesulfonyl)amino]propyl 2,4,6-trimethylbenzenesulfonate

Was prepared as described in Example 1.

### N-[(1S)-2-Amino-1-methylethyl]-2,4,6-trimethylbenzenesulfonamide

(2S)-2-[(2,4,6-Trimethylbenzenesulfonyl)amino]propyl 2,4,6-trimethylbenzenesulfonate (1 mmol, 439 mg) was dissolved in acetonitrile (3 mL) and NH₃ (32% in H₂O, 10 mL) was added. The reaction mixture stirred in room temperature for 2 hours before it was evaporated to dryness and purified on an ion exchange column (DOWEX 50WX2-400).

APCI-MS m/z: 257.1 [MH+].

### N-[(1S)-2-[[1-(4-fluorophenyl)-1H-indazol-4-yl]amino]-1-methylethyl]-2,4,6-trimethyl-benzenesulfonamide

BINAP (0.015 mmol, 9 mg) and Pd₂(dba)₃ (0.005 mmol, 5 mg) was dissolved in toluene (1 mL, 4 Å) followed by N-[(1S)-2-amino-1-methylethyl]-2,4,6-trimethylbenzenesulfonamide (0.25 mmol, 64 mg) and 4-bromo-1-(4-fluorophenyl)-1H-indazole (0.25 mmol, 73 mg) and finally sodium tert-butoxide (0.38 mmol, 36 mg). The reaction mixture was degassed and the reaction tube was filled with argon before it was heated in a microwave reactor (300 W, 15 min, 110° C.). The product was purified by silica gel column chromatography (heptane-ethyl acetate).

¹H NMR (300 MHz, DMSO-d₆) δ 8.29 (1H, s), 7.73 (2H, dd), 7.61 (1H, d), 7.40 (2H, t), 7.06 (1H, d), 6.92 (2H, s), 6.86 (1H, d), 6.47 (1H, s), 5.85 (1H, d), 3.40-2.98 (3H, m), 2.55 (6Hs), 2.17 (3H, s), 1.03 (3H, d); APCI-MS m/z: 467.1 [MH⁺].

### EXAMPLE 3

### N-((1S)-2-{[1-(6-Fluoropyridin-3-yl)-1H-indazol-4-yl]amino}-1-methylethyl)-2,4,6-trimethylbenzenesulfonamide

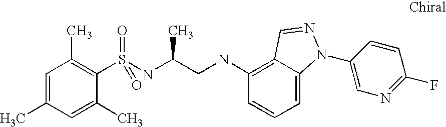

Was prepared analogous to Example 2 by use of the corresponding starting materials.

¹H NMR (300 MHz, DMSO-d₆) δ 8.61 (1H, s), 8.35 (2H, d), 7.62 (1H, d), 7.40 (1H, dd), 7.10 (1H, d), 6.94-6.87 (3H, m), 6.54 (1H, s), 5.89 (1H, d), 3.41-2.98 (3H, m), 2.55 (6H, s), 2.16 (3H, s), 1.03 (3H, d); APCI-MS m/z: 468.1 [MH⁺].

### EXAMPLE 4

### 2,4,6-Trimethyl-N-[2,2,2-trifluoro-1-({[1-(6-fluorophenyl)-1H-indazol-4-yl]oxy}methylethyl]benzenesulfonamide

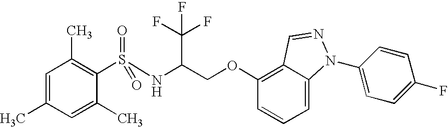

### 3-Amino-1,1,1-trifluoropropan-2-ol

2-(Trifluoromethyl)oxirane (2 g, 17.9 mmol) was stirred in aqueous ammonia (28%, 40 mL) at ambient temperature for 22 h and was then evaporated to give the title compound as a white solid (0.89 g, 38%).

¹H-NMR (300 MHz, DMSO-d₆+D₂O): 3.81 (1H, pd), 2.71 (1H, dd), 2.56 (1H, dd)

¹⁹F-NMR (282 MHz, DMSO-d₆): δ −78.00 (d)

### 2,2,2-Trifluoro-1-{[(2,4,6-trimethylbenzenesulfonyl)amino]methyl}ethyl 2,4,6-trimethylbenzenesulfonate

3-Amino-1,1,1-trifluoropropan-2-ol (1.38 g, 10.7 mmol) was dissolved in pyridine (32 mL). 2,4,6-Trimethylbenzensulfonyl chloride (7.0 g, 32 mmol) was added and the mixture was heated at reflux temperature for 18 h. After cooling, the reaction mixture was partitioned between ethyl acetate and ice water. The organic phase was washed with ice-cold saturated aqueous sodium hydrogen carbonate, twice with ice water and dried (Na₂SO₄). Chromatography (SiO₂, ethyl acetate-heptane 1:4) gave the title compound as a gum (4.4 g, 83%).

¹H-NMR (300 MHz, DMSO-d₆): 7.94 (1H, t), 7.13 (2H, s), 7.03 (2H, s), 5.00 (1H, sext), 3.27-3-16 (1H, m), 3.14-3.03 (1H, m), 2.52 (6H, s), 2.50 (6H, s), 2.30 (3H, s), 2.27 (3H, s)

¹⁹F-NMR (282 MHz, DMSO-d₆): δ −74.07 (d)

APCI-MS m/z: 494.1 [MH⁺].

### 1-(2,4,6-Trimethylbenzenesulfonyl)-2-(trifluoromethyl)aziridine

2,2,2-Trifluoro-1-{[(2,4,6-trimethylbenzenesulfonyl)amino]methyl}ethyl 2,4,6-trimethylbenzenesulfonate (4.33 g, 8.78 mmol) was dissolved in THF (190 mL). Sodium hydride (60%, 0.52 g, 13 mmol) was added in portions. The mixture was stirred at 40° C. for 15 min and then at reflux temperature for 5 h. After cooling, the mixture was partitioned between ethyl acetate and water. The organic phase was washed twice with water, once with brine and then evaporated. The crude product was combined with another batch prepared in the same way from 570 mg of 2,2,2-trifluoro-1-{[(2,4,6-trimethylbenzenesulfonyl)amino]methyl}ethyl 2,4,6-trimethylbenzenesulfonate. Chromatography (SiO₂, ethyl acetate-heptane 1:7) gave the title compound as an oil, which slowly crystallized (1.79 g, 61%).

¹H-NMR (300 MHz, CDCl₃): δ 7.01 (2H, s), 3.30-3.22 (1H, m), 2.84 (1H, d), 2.70 (6H, s), 2.50 (1H, d), 2.34 (3H, s)

¹⁹F-NMR (282 MHz, DMSO-d₆): δ −70.53 (d)

GC-MS: HP-5 column, EI at 70 EV: 293.1 [M⁺]

### 2,4,6-Trimethyl-N-[2,2,2-trifluoro-1-({[1-(6-fluorophenyl)-1H-indazol-4-yl]oxy}methyl)ethyl]benzenesulfonamide

1-(4-Fluorophenyl)-1H-indazol-4-ol (93 mg, 0.3 mmol), 1-(2,4,6-trimethylbenzenesulfonyl)-2-(trifluoromethyl)aziridine (88 mg, 0.38 mmol) and cesium carbonate (124 mg, 0.38 mmol) was stirred in dimethylformamide for 80 min. The reaction mixture was partitioned between ethyl acetate and water 1M NaOH. The organic layer was washed with 1M NaOH, brine and then evaporated. Chromatography (SiO₂, ethyl acetate-heptane 1:5) gave the title compound (60 mg, 36%).

1H-NMR (300 MHz, DMSO-d₆): δ 8.87 (1H, d), 8.04 (1H, s), 7.82-7.73 82H, m), 7.48-7.38 (2H, m), 7.36 (1H, d), 7.35 (1H, s), 6.94 (2H, s), 6.66-6.59 (1H, m), 4.55-4.39 (1H, unresolved m), 4.37-4.20 (2H, m), 2.56 (6H, s), 2.19 (3H, s).

¹⁹F-NMR (282 MHz, DMSO-d₆): δ −72.2 (d), −115.7 (tt).

APCI-MS m/z: 522.1 [MH⁺].

The following Examples were prepared analogous to Example 2 from the corresponding starting materials.

### EXAMPLE 5

### N-((1S)-2-{[1-(4-Methoxyphenyl)-1H-indazol-4-yl]amino}-1-methylethyl)-2,4,6-trimethylbenzenesulfonamide

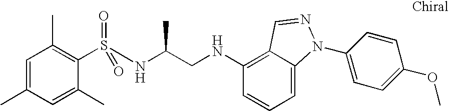

¹H NMR (400 MHz, DMSO-d₆) δ 8.24 (1H, s), 7.60 (3H, dd), 7.11 (2H, d), 7.04 (1H, t), 6.93 (2H, s), 6.79 (1H, d), 6.42 (1H, t), 5.81 (1H, d), 3.82 (3H, s), 3.41-3.31 (1H, m), 3.24-2.95 (2H, m), 2.55 (6H, s), 2.18 (3H, s), 1.03 (3H, d)

APCI-MS m/z: 479.2 [MH⁺]

### EXAMPLE 6

### 2,4,6-Trimethyl-N-[(1S)-1-methyl-2-({1-[3-(trifluoromethoxy)phenyl]-1H-indazol-4-yl}amino)ethyl]benzenesulfonamide

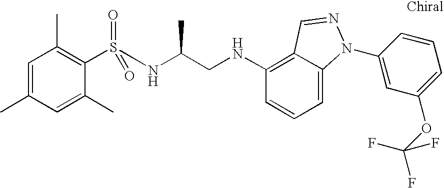

¹H NMR (400 MHz, DMSO-d₆) δ 8.35 (1H, s), 7.81 (1H, d), 7.76-7.64 (2H, m), 7.61 (1H, d), 7.35 (1H, d), 7.12 (1H, t), 6.96 (1H, d), 6.90 (2H, s), 6.53 (1H, t), 5.89 (1H, d), 3.41-3.31 (1H, m), 3.22-2.99 (2H, m), 2.54 (6H, s), 2.15 (3H, s), 1.04 (3H, d)

APCI-MS m/z: 533.2 [MH⁺]

### EXAMPLE 7

### 2,4,6-Trimethyl-N-{(1S)-1-methyl-2-[(1-phenyl-1H-indazol-4-yl)amino]ethyl}benzenesulfonamide

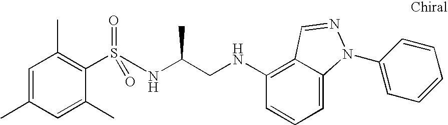

¹H NMR (400 MHz, DMSO-d₆) δ 8.29 (1H, d), 7.71 (2H, dd), 7.64-7.52 (3H, m), 7.36 (1H, t), 7.07 (1H, t), 6.93-6.89 (3H, m), 6.46 (1H, t), 5.85 (1H, d), 3.41-3.34 (1H, m), 3.20-3.01 (1H, m), 2.55 (6H, s), 2.17 (3H, s), 1.03 (3H, d)

APCI-MS m/z: 449.1 [MH⁺]

### EXAMPLE 8

### N-((1S)-2-{[1-(3-Methoxyphenyl)-1H-indazol-4-yl]amino}-1-methylethyl)-2,4,6-trimethylbenzenesulfonamide

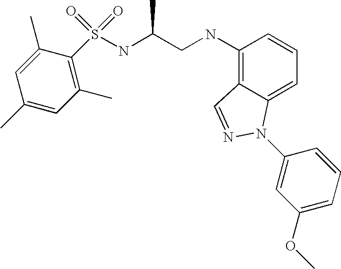

¹H NMR (400 MHz, DMSO-d₆) δ 8.29 (1H, s), 7.61 (1H, d), 7.46 (1H, t), 7.29 (1H, d), 7.23 (1H, t), 7.07 (1H, t), 6.95-6.91 (4H, m), 5.85 (1H, d), 3.84 (3H, s), 3.41-3.30 (1H, m), 3.19-2.99 (2H, m), 2.55 (6H, s), 2.17 (3H, s), 1.03 (3H, d)

APCI-MS m/z: 479.1 [MH⁺]

### EXAMPLE 9

### 2,4,6-Trimethyl-N-((1S)-1-methyl-2-{[1-(3-methylphenyl)-1H-indazol-4-yl]amino}ethyl)benzenesulfonamide

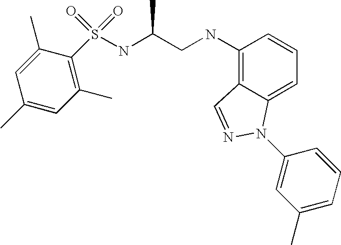

¹H NMR (400 MHz, DMSO-d₆) δ 8.28 (1H, s), 7.61 (1H, d), 7.53-7.47 (2H, m), 7.43 (1H, t), 7.17 (1H, d), 7.06 (1H, t), 6.94-6.89 (3H, m), 5.84 (1H, d), 3.40-3.31 (1H, m), 3.19-3.01 (2H, m), 2.55 (6H, s), 2.41 (3H, s), 2.18 (3H, s), 1.03 (3H, d)

APCI-MS m/z: 463.1 [MH⁺]

### EXAMPLE 10

### N-((1S)-2-{[1-(2-Fluoropyridin-4-yl)-1H-indazol-4-yl]amino}-1-methylethyl)-2,4,6-trimethylbenzenesulfonamide

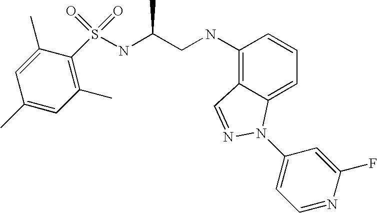

¹H NMR (400 MHz, DMSO-d₆) δ 8.45 (1H, s), 8.33 (1H, d), 7.84 (1H, d), 7.60 (1H, d), 7.53 (1H, d), 7.20-7.17 (2H, m), 6.86 (2H, s), 6.02-5.96 (1H, m), 3.35 (1H, q), 3.19-3.02 (2H, m), 2.52 (6H, s), 2.12 (3H, s), 1.05 (3H, d)

APCI-MS m/z: 468.0 [MH⁺]

### EXAMPLE 11

### N-((1S)-2-{[1-(6-Methoxypyridin-3-yl)-1H-indazol-4-yl]amino}-1-methylethyl)-2,4,6-trimethylbenzenesulfonamide

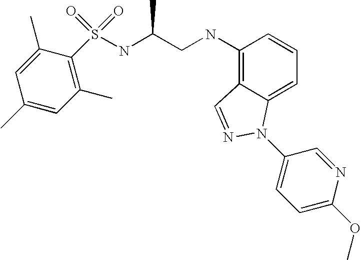

¹H NMR (400 MHz, DMSO-d₆) δ 8.49 (1H, d), 8.30 (1H, s), 8.02 (1H, dd), 7.61 (1H, d), 7.11-6.99 (2H, m), 6.93 (2H, s), 6.79 (1H, d), 5.84 (1H, d), 3.93 (3H, s), 3.40-3.28 (1H, m), 3.20-3.00 (2H, m), 2.55 (6H, s), 2.18 (3H, s), 1.03 (3H, d)

APCI-MS m/z: 480.1 [MH⁺]

### EXAMPLE 12

### 2,4,6-Trimethyl-N-((1S)-1-methyl-2-{[1-(4-methylphenyl)-1H-indazol-4-yl]amino}ethyl)benzenesulfonamide

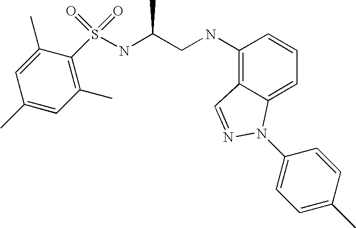

¹H NMR (400 MHz, DMSO-d₆) δ 8.26 (1H, s), 7.64-7.53 (3H, m), 7.36 (2H, d), 7.05 (1H, t), 6.92 (2H, s), 6.86 (1H, d), 5.83 (1H, d), 3.36 (1H, dd), 3.20-2.99 (2H, m), 2.55 (6H, s), 2.37 (3H, s), 2.17 (3H, s), 1.03 (3H, d)

APCI-MS m/z: 463.1 [MH⁺]

### EXAMPLE 13

### N-((1S)-2-{[1-(3-Fluorophenyl)-1H-indazol-4-yl]amino}-1-methylethyl)-2,4,6-trimethylbenzenesulfonamide

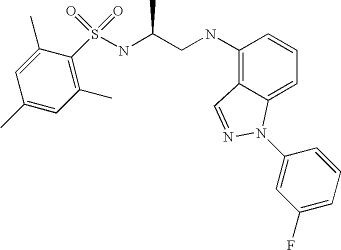

¹H NMR (400 MHz, DMSO-d₆) δ 8.33 (1H, s), 7.63-7.52 (4H, m), 7.19 (1H, quintetd), 7.10 (1H, t), 6.97 (1H, d), 6.91 (2H, s), 6.50 (1H, s), 5.88 (1H, d), 3.36 (1H, dd), 3.19-3.01 (2H, m), 2.55 (6H, s), 2.16 (3H, s), 1.04 (3H, d)

APCI-MS m/z: 467.1 [MH⁺]

### EXAMPLE 14

### 2,4,6-Trimethyl-N-{(1S)-1-methyl-2-[(1-pyridin-4-yl-1H-indazol-4-yl)amino]ethyl}benzenesulfonamide

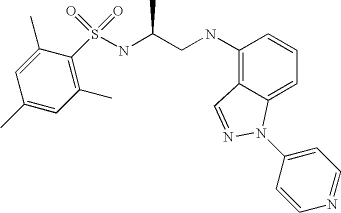

¹H NMR (400 MHz, DMSO-d₆) δ 8.79 (2H, d), 8.53 (1H, s), 8.16 (2H, d), 7.61 (1H, d), 7.25 (2H, d), 6.84 (2H, s), 6.70 (1H, s), 6.07 (1H, dd), 3.37 (1H, t), 3.19-3.05 (2H, m), 2.52 (6H, s), 2.10 (3H, s), 1.05 (3H, d)

APCI-MS m/z: 450.1 [MH⁺]

### EXAMPLE 15

### 2,4,6-Trimethyl-N-{(1S)-1-methyl-2-[(1-pyrimidin-5-yl-1H-indazol-4-yl)amino]ethyl}benzenesulfonamide

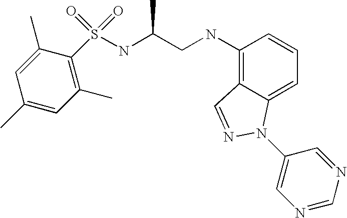

¹H NMR (400 MHz, DMSO-d₆) δ 9.26 (2H, s), 9.17 (1H, s), 8.44 (1H, s), 7.61 (1H, d), 7.14 (1H, t), 7.05 (1H, d), 6.91 (2H, s), 5.93 (1H, d), 3.36 (1H, t), 3.20-3.02 (2H, m), 2.54 (6H, s), 2.15 (3H, s), 1.04 (3H, d)

APCI-MS m/z: 451.3 [MH⁺]

### EXAMPLE 16

### 2,4,6-Trimethyl-N-{(1S)-1-methyl-2-[(1-pyridin-3-yl-1H-indazol-4-yl)amino]ethyl}benzenesulfonamide

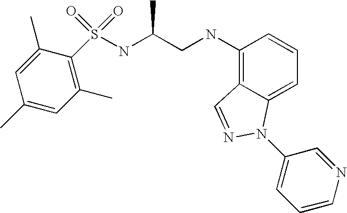

¹H NMR (400 MHz, DMSO-d₆) δ 9.03 (1H, d), 8.59 (1H, dd), 8.39 (1H, s), 8.25 (1H, dt), 7.67 (1H, dd), 7.62 (1H, d), 7.12 (1H, t), 6.97 (1H, d), 6.91 (2H, s), 5.90 (1H, d), 3.40-3.32 (1H, m), 3.20-3.02 (2H, m), 2.55 (6H, s), 2.16 (3H, s), 1.04 (3H, d)

APCI-MS m/z: 450.4 [MH⁺]

### EXAMPLE 17

### N-((1S)-2-{[1-(4-Fluoro-3-methylphenyl)-1H-indazol-4-yl]amino}-1-methylethyl)-2,4,6-trimethylbenzenesulfonamide

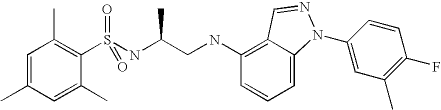

¹H NMR (400 MHz, DMSO-d₆) δ 8.27 (1H, s), 7.61 (2H, d), 7.56-7.48 (1H, m), 7.32 (1H, t), 7.07 (1H, t), 6.93 (2H, s), 6.87 (1H, d), 6.45 (1H, s), 5.84 (1H, d), 3.44-3.31 (1H, m), 3.20-3.00 (2H, m), 2.55 (6H, s), 2.34 (3H, s), 2.18 (3H, s), 1.03 (3H, d)

APCI-MS m/z: 481.1 [MH⁺]

### EXAMPLE 18

### 3-[4-({(2S)-2-[(2,4,6-Trimethylbenzenesulfonyl)amino]propyl}amino)-1H-indazol-1-yl]benzoic acid

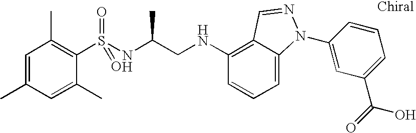

¹H NMR (400 MHz, DMSO-d₆) δ 8.27 (1H, s), 8.16 (1H, s), 7.80 (1H, d), 7.63 (1H, d), 7.57 (1H, d), 7.41 (1H, t), 7.06 (1H, t), 6.92 (2H, s), 6.88 (1H, d), 6.43 (1H, t), 5.82 (1H, d), 3.20-3.01 (2H, m), 2.55 (6H, s), 2.17 (3H, s), 1.05 (3H, d)

APCI-MS m/z: 493.1 [MH⁺]

### EXAMPLE 19

### 2,4,6-Trimethyl-N-[(1S)-1-methyl-2-({1-[3-(trifluoromethyl)phenyl]-1H-indazol-4-yl}amino)ethyl]benzene sulfonamide

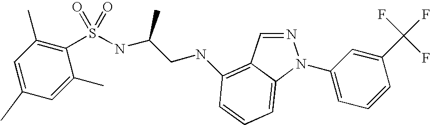

¹H NMR (400 MHz, DMSO-d₆) δ 8.50 (1H, s), 7.51 (1H, d), 7.46 (1H, t), 7.34 (2H, t), 7.18 (1H, d), 7.10 (1H, t), 6.93 (2H, s), 6.41 (1H, d), 6.00 (1H, d), 5.90 (1H, s), 3.25 (1H, quintet), 3.16-2.97 (2H, m), 2.54 (6H, s), 2.17 (3H, s), 1.00 (3H, d)

APCI-MS m/z: 517.1 [MH⁺]

### EXAMPLE 20

### N-[(1S)-2-({1-[3-(Methoxymethyl)phenyl]-1H-indazol-4-yl}amino)-1-methylethyl]-2,4,6-trimethylbenzenesulfonamide

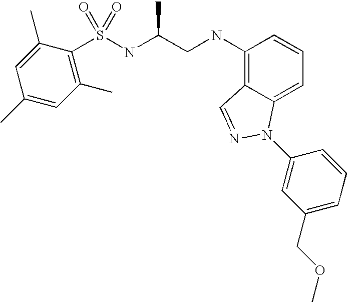

¹H NMR (400 MHz, DMSO-d₆) δ 8.29 (1H, d), 7.68-7.58 (3H, m), 7.56-7.48 (1H, m), 7.29 (1H, d), 7.08 (1H, t), 6.96-6.88 (3H, m), 6.46 (1H, t), 5.85 (1H, d), 4.53 (2H, s), 3.34 (3H, s), 3.20-2.97 (2H, m), 2.55 (6H, s), 2.17 (3H, s), 1.04 (3H, d)

APCI-MS m/z: 493.1 [MH⁺]

### EXAMPLE 21

### N-((1S)-2-{[1-(3-Fluoro-4-methoxy-phenyl)-1H-indazol-4-yl]amino}-1-methylethyl)-2,4,6-trimethylbenzenesulfonamide

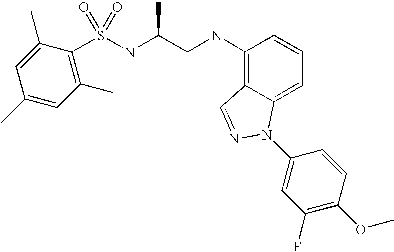

¹H NMR (400 MHz, DMSO-d₆) δ 8.27 (1H, s), 7.61 (1H, d), 7.55 (1H, dd), 7.48 (1H, d), 7.33 (1H, t), 7.07 (1H, t), 6.92 (2H, s), 6.85 (1H, d), 6.46 (1H, t), 5.84 (1H, d), 3.91 (3H, s), 3.41-3.30 (1H, m), 3.21-2.99 (2H, m), 2.55 (6H, s), 2.17 (3H, s), 1.03 (3H, d)

APCI-MS m/z: 497.1 [MH⁺]

### EXAMPLE 22

### N-((1S)-2-{[1-(4-Chlorophenyl)-1H-indazol-4-yl]amino}-1-methylethyl)-2,4,6-trimethylbenzenesulfonamide

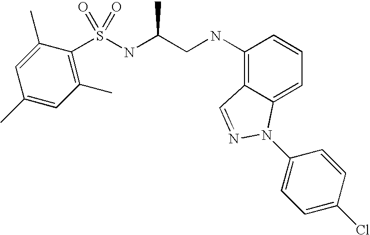

¹H NMR (400 MHz, DMSO-d₆) δ 8.31 (1H, s), 7.78-7.72 (2H, m), 7.63-7.58 (3H, m), 7.09 (1H, t), 6.95-6.87 (3H, m), 5.90-5.83 (1H, m), 3.40-3.31 (1H, m), 3.19-3.02 (2H, m), 2.54 (6H, s), 2.16 (3H, s), 1.03 (3H, d)

APCI-MS m/z: 483.1 [MH⁺]

### EXAMPLE 23

### N-((1S)-2-{[1-(4-Fluorophenyl)-5-methyl-1H-indazol-4-yl]amino}-1-methylethyl)-2,4,6-trimethylbenzenesulfonamide

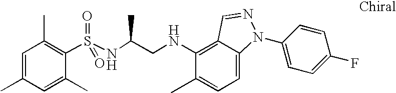

### 4-Bromo-5-methyl-1-(4-fluorophenyl)-1H-indazole

The title intermediate was prepared by use of the corresponding starting materials according to the procedure described for 4-bromo-5-fluoro-1-(4-fluorophenyl)-1H-indazole presented in Example 32.

### N-((1S)-2-{[1-(4-Fluorophenyl)-5-methyl-1H-indazol-4-yl]amino}-1-methylethyl)-2,4,6-trimethylbenzenesulfonamide

¹H NMR (400 MHz, DMSO-d₆) δ 8.25 (1H, s), 7.76-7.64 (3H, m), 7.40 (2H, t), 7.07 (1H, d), 6.92 (2H, s), 6.87 (1H, d), 5.16 (1H, d), 3.59-3.37 (3H, m), 2.51 (6H, d), 2.18 (3H, s), 2.12 (3H, s), 1.00 (3H, d)

APCI-MS m/z: 481.1 [MH⁺]

### EXAMPLE 24

### N-((2R)-2-{[1-(4-Fluorophenyl)-1H-indazol-4-yl]amino}propyl)-2,4,6-trimethylbenzenesulfonamide

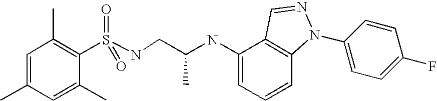

Was prepared analogous to Example 2 from the corresponding starting materials.

¹H NMR (400 MHz, DMSO-d₆) δ8.29 (1H, s), 7.73 (2H, dd), 7.61 (1H, d), 7.39 (2H, t), 7.07 (1H, t), 6.92 (2H, s), 6.86 (2H, d), 5.85 (1H, d), 3.36 (1H, t), 3.20-3.01 (2H, m), 2.55 (6H, s), 2.17 (3H, s), 1.03 (3H, d)

APCI-MS m/z: 467.1 [MH⁺]

### EXAMPLE 25

### 1-Cyclopentyl-N-((1S)-2-{[1-(4-fluoro-phenyl)-1H-indazol-4-yl]amino}-1-methylethyl-3,5-dimethyl-1H-pyrazole-4-sulfonamide

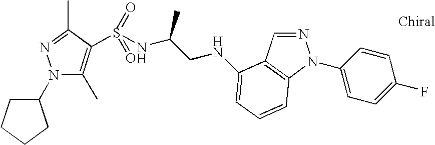

### 1-Cyclopentyl-3,5-dimethyl-1H-pyrazole-4-sulfonyl chloride

2,4-Pentadion (5.5 g, 55 mmol), cyclopentylhydrazinhydrochloride (6.83 g, 50 mmol) and DIEA (9.58 mL, 55 mmol) were dissolved in ethanol and refluxed for 48 hours. Citric acid (0.5 M) solution and ethyl acetate were added and the organic phase was washed with saturated aqueous NaHCO₃ and Brine. The organic layer was dried and evaporated to yield a colourless oil (6.70 g). The oil was dissolved in chloroform (25 mL), chilled on ice and added to chloridosulfuric acid (30 mL). The mixture was stirred at 0° C. for one hour and then refluxed for two hours. The mixture was allowed to reach room temperature, thionyl chloride (10 mL) was added and the mixture was refluxed for additional two hours. The solvents were then evaporated and the residue very slowly poured on a mixture of ice and Na₂CO₃. Water was added to the chilled neutral solution and the resulting solid (11.4 g) was collected and dried.

MS (APCI) e/z: 263.75 (MH)⁺

### N²-[(1-Cyclopentyl-3,5-dimethyl-1H-pyrazol-4-yl)sulfonyl]-L-alaninamide

1-Cyclopentyl-3,5-dimethyl-1H-pyrazole-4-sulfonyl chloride (2.62 g, 10 mmol) was dissolved in pyridine (50 mL) together with L-alaninamide hydrochloride (1.24 g, 10 mmol) and DIEA (1.7 mL, 10 mmol). The reaction mixture was stirred overnight at room temperature and then evaporated to dryness. The residue was dissolved in ethyl acetate (200 mL) and washed with 1M HCl (150 mL) and brine (150 mL). The organic phase was dried over Na₂SO₄, concentrated and used in next step without any further purification.

APCI-MS m/z: 315.1 [MH⁺]

### N-[(1S)-2-Amino-1-methylethyl]-1-cyclopentyl-3,5-dimethyl-1H-pyrazole-4-sulfonamide

N²-[(1-Cyclopentyl-3,5-dimethyl-1H-pyrazol-4-yl)sulfonyl]-L-alaninamide (crude 2.25 g, approximately 7.2 mmol) was dissolved in dry THF (5 mL) and borane-THF complex (1M, 40 mL) was added dropwise over a period of 10 min. The reaction mixture was stirred overnight at room temperature before it was quenched carefully with 1M HCl (50 mL) and diluted with ethyl acetate (150 mL). The pH of the aqueous layer was adjusted to >10 and the water phase was extracted with ethyl acetate (3×100 mL). The combined organic layers were dried, concentrated and purified by silica gel column chromatography (dichloromethane-methanol+1% NH₃).

¹H NMR (400 MHz, DMSO-d₆) δ 4.67 (1H, quintet), 3.38 (1H, dd), 2.42 (3H, s), 2.38 (2H, d), 2.25 (3H, s), 2.06-1.72 (6H, m), 1.68-1.51 (2H, m), 0.87 (3H, d)

APCI-MS m/z: 301.1 [MH⁺]

### 1-Cyclopentyl-N-((1S)-2-{[1-(4-fluorophenyl)-1H-indazol-4-yl]amino}-1-methylethyl)-3,5-dimethyl-1H-pyrazole-4-sulfonamide

Was prepared analogous to Example 2 from the corresponding starting materials.

¹H NMR (400 MHz, DMSO-d₆) δ 8.35 (1H, s), 7.72 (2H, tt), 7.46 (1H, d), 7.39 (2H, t), 7.14 (1H, t), 6.88 (1H, d), 6.51 (1H, t), 5.98 (1H, d), 4.61-4.53 (1H, m), 3.34-3.27 (1H, m), 3.25-3.16 (1H, m), 3.11-3.00 (1H, m), 2.40 (3H, d), 2.25 (3H, s), 1.97-1.67 (6H, m), 1.54 (2H, d), 1.05 (3H, d)

APCI-MS m/z: 511.2 [MH⁺]

The following Examples were prepared analogous to Example 25 by the use of the corresponding starting materials.

### EXAMPLE 26

### 1-Cyclopentyl-N-((1S)-2-{[1-(6-fluoropyridin-3-yl)-1H-indazol-4-yl]amino}-1-methylethyl)-3,5-dimethyl-1H-pyrazole-4-sulfonamide

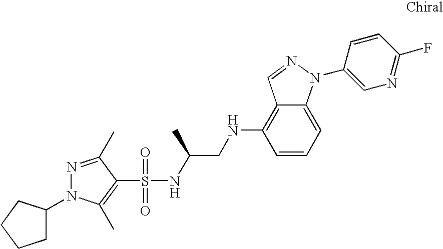

¹H NMR (400 MHz, DMSO-d₆) δ 8.60 (1H, d), 8.42 (1H, d), 8.33 (1H, ddd), 7.47 (1H, d), 7.40 (1H, dd), 7.18 (1H, t), 6.93 (1H, d), 6.58 (1H, t), 6.01 (1H, d), 4.61-4.53 (1H, m), 3.36-3.27 (1H, m), 3.26-3.17 (1H, m), 3.11-3.01 (1H, m), 2.39 (3H, s), 2.25 (3H, s), 1.97-1.67 (6H, m), 1.62-1.47 (2H, m), 1.04 (3H, d)

APCI-MS m/z: 512.2 [MH⁺]

### EXAMPLE 27

### 1-Cyclopentyl-3,5-dimethyl-N-[(1S)-1-methyl-2-({1-[4-(trifluoromethoxy)phenyl]-1H-indazol-4-yl}amino)ethyl]-1H-pyrazole-4-sulfonamide

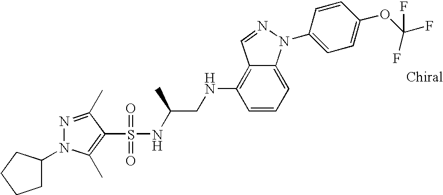

¹H NMR (400 MHz, DMSO-d₆) δ 8.39 (1H, d), 7.85 (2H, d), 7.56 (2H, d), 7.46 (1H, d), 7.17 (1H, t), 6.97 (1H, d), 6.55 (1H, t), 6.00 (1H, d), 4.61-4.52 (1H, m), 3.32-3.27 (1H, m), 3.25-3.16 (1H, m), 3.11-3.01 (1H, m), 2.39 (3H, s), 2.25 (3H, s), 1.98-1.67 (6H, m), 1.62-1.47 (2H, m), 1.05 (3H, d)

APCI-MS m/z: 577.2 [MH⁺]

### EXAMPLE 28

### 1-Cyclopentyl-N-((1S)-2-{[1-(2-methoxypyrimidin-5-yl)-1H-indazol-4-yl]amino}-1-methylethyl)-3,5-dimethyl-1H-pyrazole-4-sulfonamide

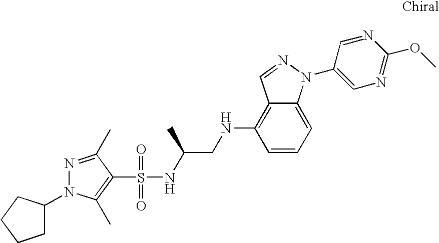

¹H NMR (400 MHz, DMSO-d₆) δ 8.97 (2H, s), 8.42 (1H, s), 7.47 (1H, d), 7.17 (1H, t), 6.88 (1H, d), 6.58 (1H, t), 6.00 (1H, d), 4.59 (1H, quintet), 4.00 (3H, s), 3.32-3.26 (1H, m), 3.26-3.17 (1H, m), 3.10-3.00 (1H, m), 2.40 (3H, s), 2.25 (3H, s), 1.99-1.67 (6H, m), 1.64-1.47 (2H, m), 1.04 (3H, d)

APCI-MS m/z: 525.3 [MH⁺]

### EXAMPLE 29

### 1-Cyclopentyl-3,5-dimethyl-N-{(1S)-1-methyl-2-[(1-pyrimidin-5-yl-1H-indazol-4-yl)amino]ethyl}-1H-pyrazole-4-sulfonamide

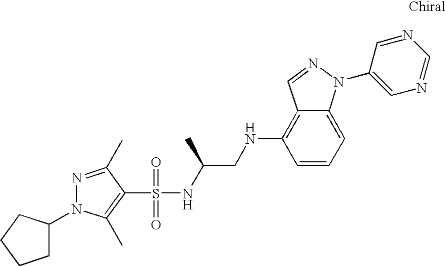

¹H NMR (400 MHz, DMSO-d₆) δ 9.25 (2H, s), 9.17 (1H, s), 8.51 (1H, s), 7.47 (1H, d), 7.22 (1H, t), 7.09 (1H, d), 6.64 (1H, t), 6.05 (1H, d), 4.58 (1H, quintet), 3.33-3.27 (1H, m), 3.26-3.17 (1H, m), 3.12-3.02 (1H, m), 2.39 (3H, s), 2.25 (3H, s), 1.98-1.65 (6H, m), 1.61-1.46 (2H, m), 1.05 (3H, d)

APCI-MS m/z: 495.3 [MH⁺]

### EXAMPLE 30

### N-((1S)-2-{[1-(4-Cyanophenyl)-1H-indazol-4-yl]amino}-1-methylethyl)-1-cyclopentyl-3,5-dimethyl-1H-pyrazole-4-sulfonamide

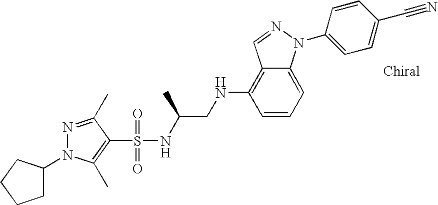

¹H NMR (400 MHz, DMSO-d₆) δ8.47 (1H, s), 8.02-7.95 (4H, m), 7.46 (1H, s), 7.22 (1H, t), 7.08 (1H, d), 6.62 (1H, t), 6.06 (1H, d), 4.56 (1H, quintet), 3.33-3.26 (1H, m), 3.26-3.17 (1H, m), 3.12-3.02 (1H, m), 2.39 (3H, s), 2.25 (3H, s), 1.97-1.66 (6H, m), 1.61-1.45 (2H, m), 1.09-1.00 (3H, m)

APCI-MS m/z: 518.3 [MH⁺]

### EXAMPLE 31

### 1-Cyclopentyl-N-((1S)-2-{[1-(5-methoxypyridin-3-yl)-1H-indazol-4-yl]amino}-1-methylethyl)-3,5-dimethyl-1H-pyrazole-4-sulfonamide

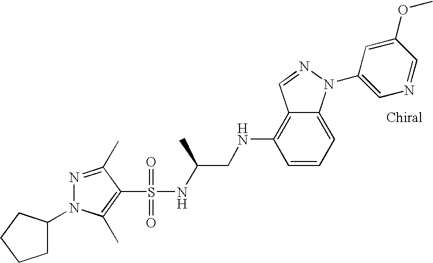

¹H NMR (400 MHz, DMSO-d₆) δ8.58 (1H, d), 8.43 (1H, d), 8.30 (1H, d), 7.67 (1H, t), 7.46 (1H, d), 7.19 (1H, t), 7.01 (1H, d), 6.58 (1H, t), 6.02 (1H, d), 4.58 (1H, t), 3.33-3.27 (1H, m), 3.25-3.16 (1H, m), 3.11-3.00 (1H, m), 2.40 (3H, s), 2.25 (3H, s), 1.99 (3H, s), 1.96-1.68 (6H, m), 1.63-1.47 (2H, m), 1.05 (3H, d)

APCI-MS m/z: 524.3 [MH⁺]

### EXAMPLE 32

### N-((1S)-2-{[5-Fluoro-1-(4-fluorophenyl)-1H-indazol-4-yl]amino}-1-methylethyl)-2,4,6-trimethylbenzenesulfonamide

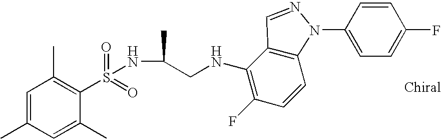

### 2-Bromo-3,6-difluorobenzaldehyde

LDA (19 mL, 29 mmol) was added dropwise to the solution of 1-bromo-2,5-difluorobenzene (5 g, 26 mmol) in THF (50 mL) at −70° C. An orange precipitate was formed. The mixture was stirred for 30 min at −75° C., and then DMF (2.0 mL, 26 mmol) was added dropwise, maintaining the temperature at −70° C. The resultant purple solution was stirred for 30 min at −70° C. and hydrolysed with dilute H₂SO₄. The organic phase was separated. The water phase was extracted with ether and the combined organic phases were evaporated. The crude product was purified by silica gel column chromatography using petroleum ether/ethyl acetate as an eluent to give the title compound (2.8 g).

GC m/z: 218/219/220/221[M].

### 4-Bromo-5-fluoro-1-(4-fluorophenyl)-1H-indazole

2-Bromo-3,6-difluorobenzaldehyde (2.8 g, 13 mmol) and 4-fluorophenylhydrazine hydrochloride (2.1 g, 13 mmol) was stirred in NMP (25 mL). Cesium carbonate (13 g, 39 mmol) was added and the reaction mixture was heated to 100° C. and stirred for 2 h. The reaction mixture was diluted with ethyl acetate, the organic phase separated and washed with diluted aqueous HCl. The water phase was extracted with ethyl acetate two times, the combined organic phases were dried over magnesium sulphate and then evaporated. The crude product was purified by silica gel column chromatography using petroleum ether/ethyl acetate as an eluent. The product was further purified by HPLC-C₁₈ to give the title compound (900 mg).

¹H NMR (400 MHz, CDCl₃): δ 8.22 (1H, d), 7.67-7.63 (2H), 7.54 (1H, m), 7.28-7.23 (3H).

APCI-MS m/z: 309, 311 [MH+].

### N-((1S)-2-{[5-Fluoro-1-(4-fluorophenyl)-1H-indazol-4-yl]amino}-1-methylethyl)-2,4,6-trimethylbenzenesulfonamide

The title compound was obtained from N-[(1S)-2-amino-1-methylethyl]-2,4,6-trimethylbenzenesulfonamide and 4-bromo-5-fluoro-1-(4-fluorophenyl)-1H-indazole by a method analogous to that described in Example 2 with the exception that the product was further purified through recrystallization from ethyl acetate and heptane.

¹H NMR (400 MHz, DMSO-d₆): δ 8.31 (1H, s), 7.74-7.70 (2H, m), 7.54 (1H, d), 7.44-7.39 (2H, m), 7.13 (1H, dd), 6.83-6.80 (3H, m), 5.85 (1H, bs) 3.41-3.31 (3H, m), 2.49 (6H, s), 2.10 (3H, s), 1.02 (3H, d).

APCI-MS m/z: 485 [MH⁺].

### EXAMPLE 33

### N-((1S)-2-{[7-Fluoro-1-(4-fluoro-phenyl)-1H-indazol-4-yl]amino}-1-methylethyl)-2,4,6-trimethylbenzenesulfonamide

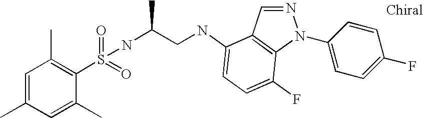

### 6-Bromo-2,3-difluorobenzaldehyde

The title compound was obtained from 1-bromo-3,4-difluorobenzene by a method analogous to that described in Example 32.

GC m/z: 218/219/220/221 [M].

### 4-Bromo-7-fluoro-1-(4-fluorophenyl)-1H-indazole

The title compound was obtained from 6-bromo-2,3-difluorobenzaldehyde and 4-fluorophenylhydrazine hydrochloride by a method analogous to that described in Example 32 with the exception that it was purified by recrystallization from methanol instead of preparative HPLC.

¹H NMR (400 MHz, CDCl₃): δ 8.23 (1H, d), 7.01-7.56 (2H), 7.29 (1H, dd), 7.23-7.18 (2H), 7.02 (1H, dd).

APCI-MS m/z: 309, 311 [MH⁺].

### N-((1S)-2-{[7-Fluoro-1-(4-fluorophenyl)-1H-indazol-4-yl]amino}-1-methylethyl)-2,4,6-trimethylbenzenesulfonamide

The title compound was obtained from N-[(1S)-2-amino-1-methylethyl]-2,4,6-trimethylbenzenesulfonamide and 4-bromo-7-fluoro-1-(4-fluorophenyl)-1H-indazole by a method analogous to that described in Example 2.

¹H NMR (400 MHz, aceton-d₆): δ 8.79 (1H, d), 7.68-7.64 (2H, m), 7.33-7.29 (2H, m), 7.19 (1H, m), 6.91-6.86 (2H, m), 6.48 (1H, d), 5.92 (1H, dd), 5.72 (1H, bs), 3.59 (1H, m), 3.27 (2H, t) 2.60 (6H, s), 2.19 (3H, s), 1.20 (3H, d). APCI-MS m/z: 485 [MH⁺].

### EXAMPLE 34

### 2,4,6-Trimethyl-N-{(1S)-1-methyl-2-[(1-phenyl-1H-pyrazolo[3,4-d]pyrimidin-4-yl)amino]ethyl}benzenesulfonamide

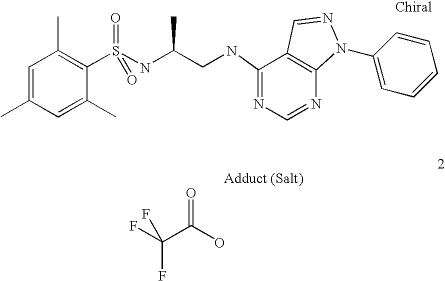

(2S)-2-[(2,4,6-Trimethylbenzenesulfonyl)amino]propyl 2,4,6-trimethylbenzenesulfonate (416 mg, 0.95 mmol) prepared as in Example 1 was dissolved in acetonitrile (4 mL). 4-Amino-1-phenylpyrazolo[3,4-d]pyrimidine (200 mg, 0.95 mmol) was added and the reaction mixture was heated to 80° C. for 24 h. The product was repeatedly purified by HPLC-C₁₈ to give the title compound (14 mg).

¹H NMR (400 MHz, dimethylsulfoxide-d₆): δ 8.41 (1H, s), 8.28 (1H, bs), 8.20 (2H, d), 7.65 (2H, t), 7.49 (1H, t), 6.56 (2H, s), 4.15 (1H, dd), 3.94 (1H, m), 3.73 (1H, m), 2.35 (6H, s) 1.85 (3H, s), 1.25 (3H, d). APCI-MS m/z: 451 [MH⁺].

### EXAMPLE 35

### N-[(1S)-1-({[1-(4-Fluorophenyl)-1H-indazol-4-yl]amino}methyl)-2-methylpropyl]-2,4,6-trimethylbenzenesulfonamide

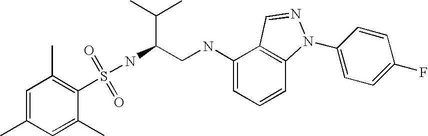

Was prepared analogous to Example 2 from the corresponding starting materials such as (S) 2-amino-3-methyl-1-butanol.

¹H NMR (400 MHz, DMSO-d₆) δ 8.25 (1H, s), 7.73 (2H, dd), 7.53 (1H, d), 7.39 (2H, t), 7.07 (1H, t), 6.94 (2H, s), 6.86 (1H, d), 6.32 (1H, s), 5.75 (1H, d), 3.26-3.16 (2H, m), 3.10-3.00 (1H, m), 2.57 (6H, s), 2.17 (3H, s), 1.90-1.80 (1H, m), 0.860 (3H, d), 0.695 (3H, d); APCI-MS m/z: 495.1 [MH+].

### EXAMPLE 36

### N-[2-[1-(4-Fluorophenyl)indazol-4-yl]sulfanyl-1-methylethyl]-2,4,6-trimethyl-benzenesulfonamide

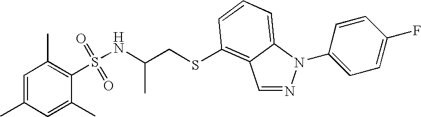

### 2,4,6-Trimethyl-N-[2-(2,4,6-trimethylphenyl)sulfonyloxypropyl]-benzenesulfonamide

The title compound was prepared by the method of Y. Yamauchi et al, Tet. Lett., 2003, 44, 6319-6322.

A mixture of 1-aminopropan-2-ol (1.56 mL, 20 mmol) and 2,4,6-trimethylbenzene-sulfonylchloride (10 g, 45.2 mmol) in pyridine (60 mL) was stirred at ambient temperature for 20 h. The reaction mixture was then evaporated and the residue partitioned between ethyl acetate and ice water. The organic phase was washed twice with ice water, once with saturated sodium hydrogen carbonate, water and finally brine. Evaporation and flash chromatography (SiO₂, heptane-ethyl acetate, gradient 0-70% heptane) gave the title compound as an oil (7.01 g, 79%), which partially crystallized when stored at −18° C.

¹H-NMR (400 MHz, DMSO-d₆): δ 7.77 (1H, t, NH); 7.09 (2H, s); 6.99 (2H, s); 4.38-4.26 (1H, m); 2.97-2.76 (2H, m); 2.48 (6H, s); 2.47 (6H, s); 2.29 (3H, s); 2.26 (3H, s); 1.07 (3H, d)

### 2-Methyl-1-(2,4,6-trimethylphenyl)sulfonylaziridine

The title compound was prepared by the method of Y. Yamauchi et al, Tet. Lett., 2003, 44, 6319-6322.

2,4,6-Trimethyl-N-[2-(2,4,6-trimethylphenyl)sulfonyloxypropyl]-benzenesulfonamide (7.01 g, 16 mmol) was dissolved in tetrahydrofuran (350 mL) under inert atmosphere. Sodium hydride (60%. 0.96 g, 24 mmol) was added in portions. After stirring at ambient temperature for 75 min, most of the solvent was evaporated at reduced pressure. Water was slowly added and the mixture was partitioned between ethyl acetate and water. The organic layer was washed twice with water, then with brine, dried (Na₂SO₄), filtered and evaporated. The crude product was pooled with a similar batch prepared from 3.0 g of 2,4,6-trimethyl-N-[2-(2,4,6-trimethylphenyl)-sulfonyloxypropyl]-benzenesulfonamide and crystallized from heptane to yield the title compound (4.62 g, 84%).

m.p. 54.5-56.0° C.

¹H-NMR (400 MHz, DMSO-d₆): δ 7.10 (2H, s); 2.81-2.68 (1H, m); 2.61 (6H, s); 2.53-2.41 (1H, m); 2.29 (3H, s); 2.15 (1H, d); 1.15 (3H, d)

APCI-MS m/z: 240.1 [MH⁺].

### N-(2-Acetylsulfanyl-1-methylethyl)-2,4,6-trimethylbenzenesulfonamide

2-Methyl-1-(2,4,6-trimethylphenyl)sulfonylaziridine (4.61 g, 19.3 mmol) was dissolved in dimethylformamide (50 mL) under inert atmosphere (Ar). Potassium thioacetate (3.2 g, 28.2 mmol) was added and the mixture was stirred at ambient temperature for 35 min and was then partitioned between ethyl acetate and water. The organic layer was washed four times with water and finally with brine. Evaporation gave crystalline title compound (5.84 g, 96%).

m.p. 123-125.5° C.

¹H-NMR (400 MHz, DMSO-d₆): 7.62 (1H, d, NH); 7.02 (2H, s); 3.28-3.12 (1H, m); 2.82 (1H, dd); 2.74 (1H, dd); 2.54 (6H, s); 2.25 (3H, s); 2.15 (3H, s); 0.99 (3H, d).

APCI-MS m/z: 316.1 [MH⁺].

### 2,4,6-Trimethyl-N-(1-methyl-2-sulfanylethyl)benzenesulfonamide

N-(2-Acetylsulfanyl-1-methylethyl)-2,4,6-trimethylbenzenesulfonamide (945 mg, 3 mmol) was dissolved in dry methanol (approximately 150 mL). The solution was degassed by evaporation to 100 mL and argon was then briefly bubbled through the clear solution. Hydrogen chloride (gaseous) was bubbled into the solution for 5 min. The reaction flask was stoppered, and the mixture stirred at ambient temperature for 16 h. Evaporation gave the title compound as off-white crystals (801 mg, 97%). m.p. 74-76° C.

¹H-NMR (400 MHz, DMSO-d₆): 7.56 (1H, d, NH); 7.03 (2H, s); 3.15 (1H, sept.); 2.56 (6H, s); 2.53-2.43 (1H, m, partially obscured by solvent signal); 2.42-2.33 (1H, m); 2.26 (3H, s); 2.20 (1H, t, SH); 0.97 (3H, d)

### N-[2-[1-(4-Fluorophenyl)indazol-4-yl]sulfanyl-1-methylethyl]-2,4,6-trimethyl-benzenesulfonamide

Sodium hydride (60%, 40 mg, 1 mmol) was added to dry N-methylpyrrolidone (1 mL) under inert atmosphere followed by a solution of 2,4,6-trimethyl-N-(1-methyl-2-sulfanylethyl)benzenesulfonamide (270 mg, 0.98 mmol) in dry N-methylpyrrolidone (1 mL). The mixture was stirred for 10 min at ambient temperature and 4-bromo-1-(4-fluorophenyl)indazole (89 mg, 0.3 mmol) was then added. The mixture was stirred at 150° C. for 1 h, then cooled and partitioned between ethyl acetate and water. The organic layer was washed four times with water and finally with brine. Evaporation and preparative HPLC gave, after lyophilisation, the title compound as its trifluoroacetic acid salt (30 mg, 16%).

¹H-NMR (400 MHz, DMSO-d₆+D₂O): 8.13 (1H, d); 7.81-7.74 (2H, m); 7.58 (1H, d); 7.49-7.40 (2H, m); 7.29 (1H, dd); 6.92 (1H, d); 6.83 (2H, s); 3.23 (1H, sext); 3.02 (1H, dd); 2.95 (1H, dd); 2.40 (6H, s); 2.11 (3H, s); 1.15 (3H, s).

¹⁹F-NMR (DMSO-d₆+D₂O): −73.70 (s); −115.22 (m).

APCI-MS m/z: 484.2 [MH⁺].

### EXAMPLE 37

### N-[2-[1-(4-Fluorophenyl)indazol-4-yl]sulfonyl-1-methyl-ethyl]-2,4,6-trimethyl-benzenesulfonamide

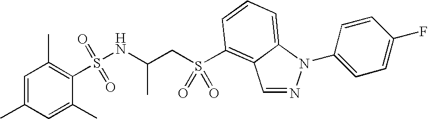

N-[2-[1-(4-Fluorophenyl)indazol-4-yl]sulfanyl-1-methyl-ethyl]-2,4,6-trimethyl-benzenesulfonamide trifluoroacetate (10.2 mg, 0.017 mmol) was dissolved in ethyl acetate (2 mL). Saturated aqueous sodium hydrogen carbonate (2 mL) was added followed by m-chloroperbenzoic acid (70%, 16 mg, 0.065 mmol). The mixture was stirred at ambient temperature for 3 h and dimethylsulfide (50 uL, 0.68 mmol) was then added to destroy excess m-chloroperbenzoic acid. Stirring was continued for 10 min and the organic layer was then separated. The aqueous layer was extracted twice with ethyl acetate and the pooled organic phases were evaporated. Preparative HPLC gave, after lyophilisation, the pure title compound as its trifluoroacetic acid salt (11 mg, quant.).

¹H-NMR (DMSO-d₆+D₂O): 8.35 (1H, s); 8.19-8.10 (1H, m); 7.87-7.76 (2H, m); 7.71-7.62 (2H, m); 7.54-7.45 (2H, m); 6.89 (2H, s); 3.51-3.39 (2H, m, partially obscured by HDO signal); 3.32-3.21 (1H, m); 2.32 (6H, s); 2.17 (3H, s); 1.17 (3H, d).

¹⁹F-NMR (DMSO-d₆+D₂O): −73.76 (s); −114.05 (m).

APCI-MS m/z: 516.2 [MH⁺].

### EXAMPLE 38

### N-{3-[1-(4-Fluorophenyl)-1H-indazol-4-yl]-1-methylpropyl}-2,4,6-trimethylbenzenesulfonamide

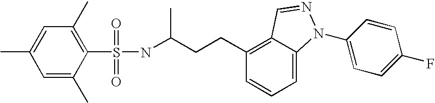

### tert-Butyl 1-methylprop-2-enylcarbamate

A solution of 2,2,2-trichloro-N-(1-methylprop-2-enyl)acetamide [prepared according to L. E. Overman, J. Am. Chem. Soc., 98, 2901-2909 (1976)] (2.75 g, 12.7 mmol) in ethanol (20 mL) and 6M aqueous sodium hydroxide (20 mL) was stirred overnight. Di-tert-butyl dicarbonate (5.54 g, 25.4 mmol) was added at 0° C., and the mixture was stirred at room temperature for 2 h. Extraction with diethyl ether, drying over magnesium sulfate and evaporation gave an oil. This was purified by chromatography (SiO₂, dichloromethane/ethyl acetate 40/1) to give the title compound as a liquid (341 mg).

¹H NMR (400 MHz, CD₂Cl₂) δ 5.82 (1H, m), 5.12 (1H, m), 5.04 (1H, m), 4.52 (1H, broad s), 4.16 (1H, broad s), 1.42 (9H, s), 1.19 (3H, d).

### tert-Butyl (2E)-3-[1-(4-fluorophenyl)-1H-indazol-4-yl]-1-methylprop-2-enylcarbamate

A mixture of tert-butyl 1-methylprop-2-enylcarbamate (255 mg, 1.49 mmol), 4-bromo-1-(4-fluorophenyl)-1H-indazole (see Example 2) (220 mg, 0.75 mmol), tetrabutylammonium iodide (415 mg, 1.12 mmol), N-ethyldiisopropylamine (1.5 mL), Pd-118 (49 mg, 0.075 mmol) and DMF (10 mL) was stirred under an argon atmosphere at 60° C. overnight. The mixture was concentrated and partitioned between water and ethyl acetate. The organic phase was washed with brine, dried over magnesium sulfate and evaporated. Purification by chromatography (SiO₂, dichloromethane/ethyl acetate 20/1) gave the title compound as an oil (165 mg).

¹H NMR (400 MHz, CD₂Cl₂) δ 8.35 (1H, m), 7.73-7.66 (2H, m), 7.57 (1H, d), 7.40 (1H, m), 7.30-7.22 (3H, m), 6.89 (1H, m), 6.46 (1H, dd), 4.70 (1H, broad s), 4.46 (1H, broad s), 1.46 (9H, s), 1.38 (3H, d).

### tert-Butyl 3-[1-(4-fluorophenyl)-1H-indazol-4-yl]-1-methylpropylcarbamate

A solution of tert-butyl (2E)-3-[1-(4-fluorophenyl)-1H-indazol-4-yl]-1-methylprop-2-enylcarbamate (165 mg, 0.43 mmol) in ethanol (20 mL) was hydrogenated over Pd on carbon (5%, 50 mg) at atmospheric pressure for 2.5 h. Filtering through Celite and evaporation gave the title compound (158 mg).

¹H NMR (400 MHz, CD₂Cl₂) δ 8.22 (1H, s), 7.73-7.66 (2H, m), 7.53 (1H, d), 7.36 (1H, m), 7.29-7.21 (2H, m), 7.05 (1H, d), 4.51 (1H, broad s), 3.74 (1H, broad s), 3.02 (2H, m), 1.89 (2H, m), 1.44 (9 h, s), 1.19 (3H, dd).

### 3-[1-(4-Fluorophenyl)-1H-indazol-4-yl]-1-methylpropylamine trifluoroacetate salt

Trifluoroacetic acid (1.2 mL) was added to a solution of tert-butyl 3-[1-(4-fluorophenyl)-1H-indazol-4-yl]-1-methylpropylcarbamate (155 mg, 0.40 mmol) in dichloromethane (6 mL). After stirring for 3 h the solution was evaporated and co-evaporated with toluene to give the title compound (203 mg).

APCI-MS m/z: 284.1 [MH⁺].

### N-(3-[1-(4-Fluorophenyl)-1H-indazol-4-yl]-1-methylpropyl)-2,4,6-trimethylbenzenesulfonamide

A solution of 2,4,6-trimethylbenzenenesulfonyl chloride (175 mg, 0.80 mmol) in THF (5 mL) was added to 3-[1-(4-fluorophenyl)-1H-indazol-4-yl]-1-methylpropylamine trifluoroacetate salt (159 mg, 0.40 mg) and N-diisopropylethylamine (0.40 mL) in THF (4 mL). The mixture was stirred overnight, concentrated and purified by chromatography (SiO₂, dichloromethane/ethyl acetate 20/1-10/1), followed by HPLC-C₁₈. Concentration and lyophilisation from tert-butanol gave the title compound (77 mg).

¹H NMR (400 MHz, CD₂Cl₂) δ 8.05 (1H, broad s), 7.69 (2H, m), 7.51 (1H, d), 7.30 (1H, dd), 7.28-7.22 (2H, m), 6.96 (2H, broad s), 6.84 (1H, d), 4.46 (1H, d), 3.35 (1H, m), 2.98-2.78 (2H, m), 2.59 (6H, s), 2.28 (3H, s), 1.86-1.75 (2H, m), 1.14 (3H, d); APCI-MS m/z: 466.2 [MH⁺].

### EXAMPLE 39

### N-{(1S)-3-[1-(4-Fluorophenyl)-1H-indazol-4-yl]-1-methylpropyl}-2,4,6-trimethylbenzenesulfonamide

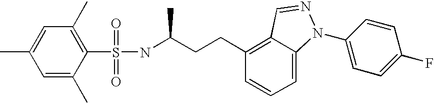

### tert-Butyl (1S)-1-methylprop-2-enylcarbamate

n-Butyl lithium (2.5 M in hexane, 19.4 mL, 48.4 mmol) was added dropwise under 15 minutes to a suspension of methyltriphenylphosphonium bromide (20.2 g, 56.6 mmol) in anhydrous THF (200 mL) at 0° C. The mixture was stirred at 0° C. for 30 minutes, then at room temperature for 11 h. The mixture was cooled to −20° C. and tert-butyl [(1S)-1-methyl-2-oxoethyl]carbamate (7.00 g, 40.0 mmol) dissolved in anhydrous THF (100 mL) was added dropwise during 1 h. After stirring overnight at room temperature and 2 h at 50° C., the mixture was cooled with an ice-bath. Saturated aqueous ammonium chloride and water was added to give a clear solution. The mixture was extracted with diethyl ether (250 mL), and the organic phase was dried over magnesium sulfate. Distillation of the solvents at atmospheric pressure, followed by vacuum distillation gave the title compound (86° C., 13 mbar) as a liquid (2.1 g).

¹H NMR (400 MHz, CD₂Cl₂) δ 5.83 (1H, m), 5.12 (1H, m), 5.04 (1H, m), 4.51 (1H, broad s), 4.17 (1H, broad s), 1.42 (9H, s), 1.19 (3H, d, J=6.9 Hz).

### Tert-Butyl (1S,2E)-3-[1-(4-fluorophenyl)-1H-indazol-4-yl]-1-methylprop-2-enylcarbamate

The title compound (163 mg) was prepared from tert-butyl (1S)-1-methylprop-2-enylcarbamate (255 mg, 1.49 mmol) and 4-bromo-1-(4-fluorophenyl)-1H-indazole (220 mg, 0.75 mmol) by a method analogous to that described in Example 38.

¹H NMR (400 MHz, CD₂Cl₂) δ 8.35 (1H, d), 7.73-7.66 (2H, m), 7.57 (1H, d), 7.40 (1H, m), 7.30-7.22 (3H, m), 6.89 (1H, m), 6.46 (1H, dd, J=5.7 Hz, J₂=16.0 Hz), 4.70 (1H, broad s), 4.46 (1H, broad s), 1.46 (9H, s), 1.38 (3H, d, J=6.8 Hz).

### Tert-Butyl (1S)-3-[1-(4-fluorophenyl)-1H-indazol-4-yl]-1-methylpropylcarbamate

The title compound (78 mg) was prepared from tert-butyl (1S,2E)-3-[1-(4-fluorophenyl)-1H-indazol-4-yl]-1-methylprop-2-enylcarbamate (78 mg, 0.20 mmol) analogously to that described in Example 38.

APCI-MS m/z: 384.1 [MH⁺].

### (1S)-3-[1-(4-Fluorophenyl)-1H-indazol-4-yl]-1-methylpropylamine

Trifluoroacetic acid (0.60 mL) was added to a solution of tert-butyl (1S)-3-[1-(4-fluorophenyl)-1H-indazol-4-yl]-1-methylpropylcarbamate (78 mg, 0.20 mmol) in dichloromethane (3.0 mL). After stirring for 1 h the solution was evaporated and co-evaporated with toluene. Conversion into the base form on a BondElut SCX ion exchange column using methanol/ammonia as eluent gave the title compound (54 mg).

APCI-MS m/z: 284.1 [MH⁺].

### N-{(1S)-3-[1-(4-Fluorophenyl)-1H-indazol-4-yl]-1-methylpropyl}-2,4,6-trimethylbenzenesulfonamide

(1S)-3-[1-(4-Fluorophenyl)-1H-indazol-4-yl]-1-methylpropylamine (54 mg, 0.19 mmol) was reacted with 2,4,6-benzenesulfonyl chloride (83 mg, 0.38 mmol) by a method analogous to that described in Example 38. Purification by HPLC-C₁₈, followed by lyophilisation from tert-butanol gave the title compound (79 mg).

¹H NMR (400 MHz, CD₂Cl₂) δ 8.05 (1H, m), 7.69 (2H, m), 7.51 (1H, d), 7.30 (1H, dd), 7.28-7.22 (2H, m), 6.96 (2H, broad s), 6.84 (1H, d), 4.46 (1H, d), 3.35 (1H, m), 2.98-2.78 (2H, m), 2.59 (6H, s), 2.28 (3H, s), 1.86-1.75 (2H, m), 1.14 (3H, d, J=6.6 Hz); APCI-MS m/z: 466.1 [MH⁺]. The enantiomeric excess was determined to be 82% (SFC, Kromasil CHI-TBB, 10% MeOH).

### EXAMPLE 40

### N-((2S)-2-{[1-(4-Fluorophenol)-1H-indazol-4-yl]amino}propyl)-2,4,6-trimethylbenzenesulfonamide

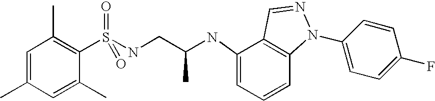

Was prepared analogous to Example 2 from the corresponding starting materials.

¹H NMR (400 MHz, DMSO) δ 8.29 (d, 1H), 7.75-7.69 (m, 2H), 7.61 (d, 1H), 7.40 (t, 2H), 7.07 (t, 1H), 6.92 (s, 2H), 6.86 (d, 1H), 6.47 (s, 1H), 5.85 (d, 1H), 3.21-3.00 (m, 3H), 2.55 (s, 6H), 2.17 (s, 3H), 1.03 (d, 3H)

APCI-MS m/z: 467.1 [MH+].

### EXAMPLE 41

### N-((1S)-2-{[1-(4-Fluorophenyl)-1H-indazol-4-yl]oxy}-1-methylethyl)-3,5-dimethyl-1H-pyrazole-4-sulfonamide

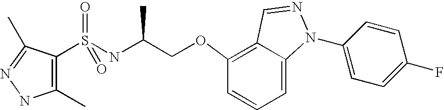

### 1-(4-Fluorophenyl)-1H-indazol-4-ol

Was prepared as described in Example 1.

### 2-[(1S)-2-Hydroxy-1-methylethyl]-1H-isoindole-1,3(2H)-dione

Phthalic anhydride (50 mmol, 7.4 g) was dissolved in 100 mL toluene together with L-alaninol (50 mmol, 3.9 mL) and DIEA (5 mmol, 900 μL). The mixture was refluxed with continuous removal of water with a Dean-Stark apparatus for two hours before it was washed with 1M HCl, saturated aqueous NaHCO₃. The organic layer was dried, concentrated and used in the next step without any further purification.

APCI-MS m/z: 206.0 [MH⁺].

### (2S)-2-(1,3-Dioxo-1,3-dihydro-2H-isoindol-2-yl)propyl 4-methylbenzenesulfonate

4-Methylbenzenesulfonyl chloride (43 mmol, 8.2 g) and 2-[(1S)-2-hydroxy-1-methylethyl]-1H-isoindole-1,3(2H)-dione (43 mmol, 8.8 g) were dissolved in pyridine (200 mL) and stirred overnight in room temperature. The mixture was evaporated, dissolved in ethyl acetate (200 mL) and washed with 1M HCl, saturated aqueous NaHCO₃. The organic layer was dried, concentrated and purified by silica gel column chromatography (heptane-ethyl acetate).

APCI-MS m/z: 360.0 [MH+].

### 2-((1S)-2-{[1-(4-Fluorophenyl)-1H-indazol-4-yl]oxy}-1-methylethyl)-1H-isoindole-1,3(2H)-dione

(2S)-2-(1,3-Dioxo-1,3-dihydro-2H-isoindol-2-yl)propyl 4-methyl-benzenesulfonate (12.1 mmol, 4.36 g) was dissolved in DMF (50 mL) together with 1-(4-fluorophenyl)-1H-indazol-4-ol (11 mmol, 2.5 g). Cesium carbonate (17 mmol, 5.5 g) was added and the reaction mixture was stirred and heated to 100° C. for 2 hours before it was evaporated, dissolved in ethyl acetate (200 mL) and washed with 1M HCl. The organic layer was dried, concentrated and purified by silica gel column chromatography (heptane-ethyl acetate).

APCI-MS m/z: 416.0 [MH+].

### ((1S)-2-{[1-(4-Fluorophenyl)-1H-indazol-4-yl]oxy}-1-methylethyl)amine

2-((1S)-2-{[1-(4-Fluorophenyl)-1H-indazol-4-yl]oxy}-1-methylethyl)-1H-isoindole-1,3(2H)-dione (6.7 mmol, 2.8 g) was dissolved in methylamine (33% in ethanol, 50 mL) and stirred overnight at room temperature. The reaction mixture was evaporated to dryness and purified by silica gel column chromatography (dichloromethane-methanol+1% NH₃).

APCI-MS m/z: 286.1 [MH+].

### N-((1S)-2-{[1-(4-Fluorophenyl)-1H-indazol-4-yl]oxy}-1-methylethyl)-3,5-dimethyl-1H-pyrazole-4-sulfonamide

3,5-Dimethyl-1H-pyrazole-4-sulfonyl chloride (1.5 mmol, 292 mg) was mixed together with ((1S)-2-{[1-(4-fluorophenyl)-1H-indazol-4-yl]oxy}-1-methylethyl)amine (1 mmol, 285 mg) and DIEA (3 mmol, 387 mg) in THF (30 mL). The reaction mixture was refluxed for 5 hours before it was diluted with ethyl acetate (150 mL) and washed with saturated aqueous NaHCO₃. The organic layer was dried, concentrated and purified by silica gel column chromatography (heptane-ethyl acetate).

¹H NMR (300 MHz, DMSO-d₆) δ 8.24 (d, 1H), 7.78 (tt, 2H), 7.66 (d, 1H), 7.46-7.29 (m, 4H), 6.59 (dd, 1H), 4.10-3.88 (m, 2H), 3.60-3.19 (m, 2H), 2.31 (s, 6H), 1.16 (d, 2H)

APCI-MS m/z: 444.0 [MH+].

### EXAMPLE 42

### 3,5-Dimethyl-N-{(1S)-1-methyl-2-[(1-pyridin-3-yl-1H-indazol-4-yl)oxy]ethyl}-1H-pyrazole-4-sulfonamide

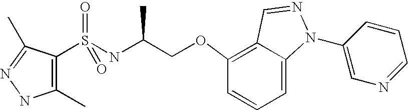

Was prepared analogous to Example 41 from the corresponding starting materials.

¹H NMR (400 MHz, DMSO-d₆) δ 9.06 (d, 1H), 8.63 (d, 1H), 8.33 (s, 1H), 8.27 (d, 1H), 7.70-7.64 (m, 2H), 7.42 (dd, 2H), 6.64 (d, 1H), 5.75 (s, 1H), 4.06 (dd, 1H), 3.94 (dd, 1H), 3.53 (dd, 1H), 2.30 (s, 6H), 1.16 (d, 3H)

APCI-MS m/z: 427.4 [MH+].

### EXAMPLE 43

### 1-tert-Butyl-N-((1S)-2-[{1-(4-fluorophenyl)-1H-indazol-4-yl]oxy}-1-methylethyl)-3,5-dimethyl-1H-pyrazole-4-sulfonamide

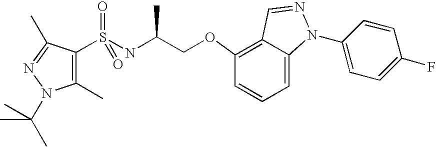

### (1S)-2-{[1-(4-Fluorophenyl)-1H-indazol-4-yl]oxy}-1-methylethyl)amine

Was prepared as described in Example 41.

### 1-tert-Butyl-3,5-dimethyl-1H-pyrazole-4-sulfonyl chloride

A solution of 1-tert-butyl-3,5-dimethyl-1H-pyrazole (6.57 mmol, 1 g) in chloroform (5 mL) was added dropwise to chlorosulfonic acid (approximately 66 mmol, 4.5 mL) cooled to 0° C. After addition temperature was slowly raised to 40° C. and the reaction mixture was stirred for 2 hours before thionyl chloride (approximately 28 mmol, 2 mL) was added dropwise. The mixture was stirred at 40° C. for another 4 hours before excess of reagents was removed by evaporation and the reaction was quenched by dropwise adding it to a ice/water slurry (200 mL). The product was extracted with chloroform (2×100 mL) and the combined organic layers were dried, concentrated and purified by silica gel column chromatography (heptane-ethyl acetate).

### 1-tert-Butyl-N-((1S)-2-[1-(4-fluorophenyl)-1H-indazol-4-yl]ox-1-methylethyl)-3,5-dimethyl-1H-pyrazole-4-sulfonamide

The sulfonamide was prepared as described in Example 41 from the corresponding starting materials.

¹H NMR (400 MHz, DMSO-d₆) δ 8.22 (s, 1H), 7.70 (dd, 2H), 7.65 (d, 1H), 7.37 (t, 2H), 7.28 (dd, 2H), 6.49 (d, 1H), 3.95 (dd, 1H), 3.81 (dd, 1H), 3.49-3.40 (m, 1H), 2.50 (s, 3H), 2.20 (s, 3H), 1.39 (s, 9H), 1.13 (d, 3H)

APCI-MS m/z: 500.5 [MH+].

### EXAMPLE 44

### 1-tert-Butyl-3,5-dimethyl-N-{(1S)-1-methyl-2-[(1-pyridin-3-yl-1H-indazol-4-yl)oxy]ethyl}-1H-pyrazole-4-sulfonamide

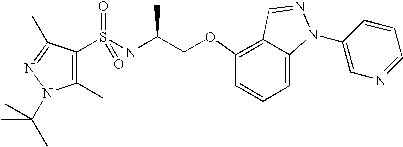

Was prepared analogous to Example 43 by the use of the corresponding starting material.

¹H NMR (400 MHz, DMSO-d₆) δ 9.03 (d, 1H), 8.62 (d, 1H), 8.37 (s, 1H), 8.23 (d, 1H), 7.72 (d, 1H), 7.66 (dd, 1H), 7.41 (dd, 2H), 6.60 (d, 1H), 4.02 (dd, 1H), 3.88 (dd, 1H), 3.60-3.51 (m, 1H), 2.56 (s, 3H), 2.25 (s, 3H), 1.45 (s, 9H), 1.19 (d, 3H)

APCI-MS m/z: 483.5 [MH+].

### EXAMPLE 45

### N-((1S)-2-{[1-(4-Fluorophenyl)-1H-indazol-4-yl]oxy}-1-methylethyl)-3,5-dimethyl-1-[(3R)-tetrahydrofuran-3-yl]-1H-pyrazole-4-sulfonamide

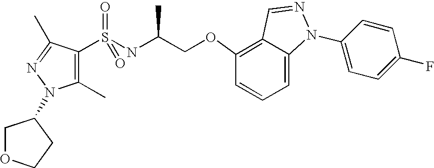

### (3R)-Tetrahydrofuran-3-yl 4-methylbenzenesulfonate

(3R)-Tetrahydrofuran-3-ol (20 mmol, 1.76 g) was dissolved in 100 mL pyridine together with 4-methylbenzenesulfonyl chloride (21 mmol, 4 g) and stirred overnight at room temperature. Solvent was removed by evaporation and the residue was dissolved in dichloromethane (100 mL) and washed with 1M HCl, saturated aqueous NaHCO₃. The organic layer was dried, concentrated and used in the next step without any further purification.

### N-((1S)-2-{[1-(4-Fluorophenyl)-1H-indazol-4-yl]oxy}-1-methylethyl)-3,5-dimethyl-1H-pyrazole-4-sulfonamide

Was prepared as described in Example 41.

### N-((1S)-2-{[1-(4-Fluorophenyl)-1H-indazol-4-yl]oxy}-1-methylethyl)-3,5-dimethyl-1-[(3R)-tetrahydrofuran-3-yl]-1H-pyrazole-4-sulfonamide

N-((1S)-2-{[1-(4-Fluorophenyl)-1H-indazol-4-yl]oxy}-1-methylethyl)-3,5-dimethyl-1H-pyrazole-4-sulfonamide (0.15 mmol, 66 mg) was mixed with (3R)-tetrahydrofuran-3-yl 4-methylbenzenesulfonate (0.20 mmol, 48 mg) in butyronitrile together with cesium carbonate (0.5 mmol, 162 mg). The reaction mixture was stirred and heated at 120° C. for 2 hours. Solvent was removed by evaporation and the residue was dissolved in dichloromethane (20 mL) and washed with 1M HCl. The organic layer was dried, concentrated and the residue purified by HPLC-C₁₈.

¹H NMR (400 MHz, DMSO-d₆) δ 8.24 (s, 1H), 7.83-7.69 (m, 3H), 7.43 (t, 2H), 7.34 (dd, 2H), 6.57 (d, 1H), 4.92 (septet, 1H), 4.02 (dd, 1H), 3.94-3.86 (m, 3H), 3.76 (td, 1H), 3.65 (dd, 1H), 3.56 (dd, 1H), 2.43 (s, 3H), 2.27 (s, 3H), 2.26-2.15 (m, 1H), 2.10 (ddd, 1H), 1.18 (d, 3H)

APCI-MS m/z: 514.4 [MH+].

The following Examples were prepared analoguesly to Example 45 by the use of the corresponding starting materials.

### EXAMPLE 46

### 1-(1-Ethylpropyl)-N-((1S)-2-{[1-(4-fluorophenyl)-1H-indazol-4-yl]oxy}-1-methylethyl)-3,5-dimethyl-1H-pyrazole-4-sulfonamide

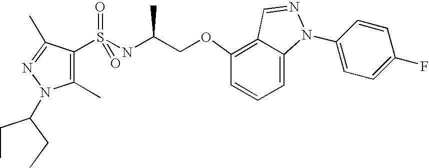

¹H NMR (400 MHz, DMSO-d₆) δ 8.31 (s, 1H), 7.77 (dd, 2H), 7.71 (d, 1H), 7.42 (t, 2H), 7.34 (dd, 2H), 6.59 (d, 1H), 4.01 (ddd, 2H), 3.90 (dd, 1H), 3.51 (dd, 1H), 2.43 (s, 3H), 2.30 (s, 3H), 1.83-1.62 (m, 4H), 1.14 (d, 3H), 0.59 (t, 6H)

APCI-MS m/z: 514.5 [MH+].

### EXAMPLE 47

### N-((1S)-2-{[1-(4-Fluorophenyl)-1H-indazol-4-yl]oxy}-1-methylethyl)-3,5-dimethyl-1-[(3S)-tetrahydrofuran-3-yl]-1H-pyrazole-4-sulfonamide

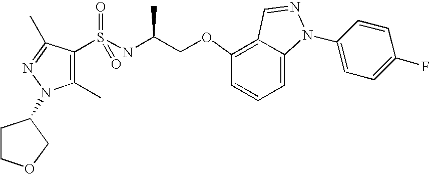

¹H NMR (400 MHz, DMSO-d₆) δ 8.23 (s, 1H), 7.82-7.67 (m, 3H), 7.43 (t, 2H), 7.34 (dd, 2H), 6.57 (d, 1H), 4.91 (septet, 1H), 4.02 (dd, 1H), 3.97-3.86 (m, 3H), 3.76 (dd, 1H), 3.69 (dd, 1H), 3.60-3.51 (m, 1H), 2.43 (s, 3H), 2.27 (s, 3H), 2.23-2.13 (m, 1H), 2.12-2.00 (m, 1H), 1.18 (d, 3H)

APCI-MS m/z: 514.4 [MH+].

### EXAMPLE 48

### 1-Cyclopentyl-N-((1S)-2-{[1-(4-fluorophenyl)-1H-indazol-4-yl]oxy}-1-methylethyl)-3,5-dimethyl-1H-pyrazole-4-sulfonamide

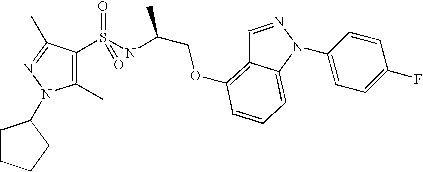

¹H NMR (400 MHz, DMSO-d₆) δ 8.25 (s, 1H), 7.77 (dd, 2H), 7.68 (d, 1H), 7.46-7.40 (m, 2H), 7.34 (dd, 2H), 6.56 (d, 1H), 4.59 (t, 1H), 4.02 (dd, 1H), 3.90 (dd, 1H), 3.59-3.52 (m, 1H), 2.41 (s, 3H), 2.27 (s, 3H), 1.98-1.68 (m, 6H), 1.55 (s, 2H), 1.18 (d, 3H)

APCI-MS m/z: 512.1 [MH+].

### EXAMPLE 49

### 1-Cyclopentyl-3,5-dimethyl-N-{(1S)-1-methyl-2-[(1-pyridin-3-yl-1H-indazol-4-yl)oxy]ethyl}-1H-pyrazole-4-sulfonamide

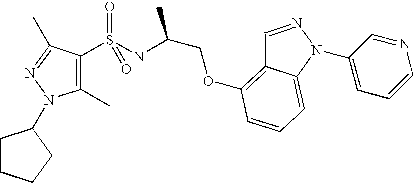

¹H NMR (400 MHz, DMSO-d₆) δ 9.03 (d, 1H), 8.62 (d, 1H), 8.33 (s, 1H), 8.22 (d, 1H), 7.69 (d, 1H), 7.65 (dd, 1H), 7.41 (dd, 2H), 6.61 (d, 1H), 4.58 (t, 1H), 4.03 (dd, 1H), 3.91 (dd, 1H), 3.61-3.50 (m, 1H), 2.41 (s, 3H), 2.27 (s, 3H), 1.99-1.84 (m, 2H), 1.84-1.69 (m, 4H), 1.64-1.47 (m, 2H), 1.18 (d, 3H)

APCI-MS m/z: 495.1 [MH+].

### EXAMPLE 50

### N-{(1S)-2-[(1-Cyclopentyl-1H-indazol-4-yl)amino]-1-methylethyl}-2,4,6-trimethylbenzene sulfonamide

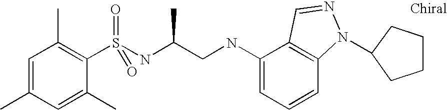

### Cyclopentylhydrazine:

The compound was prepared in three steps according to the method described by Ramani R. Ranatunge et al J. Med. Chem., 2004, 47, 2180-2193.

### 4-Bromo-1-cyclopentyl-1H-indazole

The title compound was obtained from 2-bromo-6-fluorobenzaldehyde and cyclopentylhydrazine trifluoroacetate by a method analogously to that described in Example 32 with the exception that the reaction mixture was heated in a microwave reactor (200 W, 50 min, 100° C.).

APCI-MS m/z: 265 [MH+].

### N-{(1S)-2-[(1-Cyclopentyl-1H-indazol-4-yl)amino]-1-methylethyl}-2,4,6-trimethylbenzenesulfonamide

The title compound was obtained from N-[(1S)-2-Amino-1-methylethyl]-2,4,6-trimethylbenzenesulfonamide and 4-bromo-1-cyclopentyl-1H-indazole by a method analogous to that described in Example 2 with the exception that the product was further purified by HPLC-C₁₈ to give the title compound.

¹H NMR (400 MHz, DMSO-d₆): δ 7.98 (1H, s), 7.60 (1H, bs), 6.96-6.92 (3H), 6.73 (1H, d), 6.24 (1H, t), 5.69 (1H, d), 4.96 (1H, m), 3.30 (1H), 3.10 (1H, m), 3.00 (1H, m), 2.55 (6H, s), 2.22 (3H, s), 2.09-1.80 (6H), 1.70-1.63 (2H), 0.98 (3H, d). APCI-MS m/z: 440 [MH+].

### EXAMPLE 51

### N-((1S)-1-Ethyl-2-{[1-(4-methylphenyl)-1H-indazol-4-yl]amino}ethyl)benzenesulfonamide

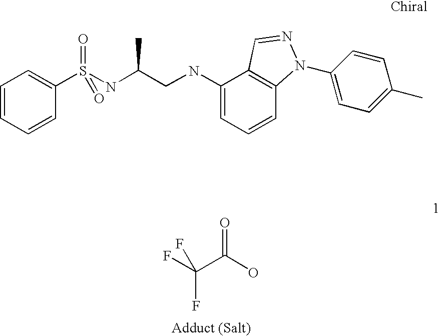

### N-[(1S)-2-Amino-1-methylethyl]-benzenesulfonamide

The title compound was obtained from L-alaninol and benzenesulphonyl chloride by a method analogous to that described in Example 2.

¹H NMR (400 MHz, DMSO-d₆): δ 7.81 (2H, m), 7.59 (3H, m), 3.02 (1H, m), 2.38 (2H, m), 0.85 (3H, d). APCI-MS m/z: 215 [MH⁺].

### N-((1S)-1-Methyl-2-{[1-(4-methylphenyl)-1H-indazol-4-yl]amino}ethyl)benzenesulfonamide

The title compound was obtained from N-[(1S)-2-amino-1-methylethyl]-benzenesulfonamide and 4-bromo-1-(4-methylphenyl)-1H-indazole by a method analogous to that described in Example 2 with the exception that the reaction mixture was stirred for 24 h at 90° C. in an oil bath and the final product was further purified by HPLC-C₁₈ to give the title compound.

¹H NMR (400 MHz, DMSO-d₆): δ 8.30 (1H, d), 7.82-7.78 (3H), 7.59-7.45 (5H), 7.38-7.34 (2H), 7.10 (1H, t), 6.86 (1H, d), 6.45 (1H, bt) 5.99 (1H, d), 3.42-3.20 (2H), 3.12-3.03 (1H, m), 2.37 (3H, s), 1.01 (3H, s). APCI-MS m/z: 420 [MH+].

### EXAMPLE 52

### N-((1S)-2-{[1-(4-Fluoro-phenyl)-3-methyl-1H-indazol-4-yl]amino}-1-methylethyl)-2,4,6-trimethylbenzenesulfonamide

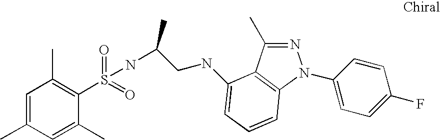

### (2-Bromo-6-fluorophenyl)-(trimethyl)silane

The compound was prepared according to the method described by Sergiusz Lulinski et al J. Org. Chem. 2003, 68, 9384-9388. 1-Bromo-3-fluoro-benzene (28.6 mmol, 5.0 g) and trimethylsilylchloride (34.3 mmol, 3.73 mg) was dissolved in THF (40 mL) and lithium diisopropylamide (17 mL, 2M) was added dropwise at −70° C. The reaction mixture was stirred for 1 h and then hydrolyzed with dilute aqueous sulphuric acid. The organic phase was separated, the water phase extracted with ether and the combined organic phases were evaporated. The crude product was distilled bp 82-94° C. (10 mm Hg) to give the title compound (3.61 g).

¹H NMR (400 MHz, CDCl₃): δ 7.35 (1H, dd), 7.16 (1H, m), 6.94 (1H, m), 0.46 (9H, d).

### 1-(2-Bromo-6-fluorophenyl)ethanone

The compound was prepared according to the method described by Bernard Bennetau et al, Tetrahedron Vol 49, No. 47, pp 10843-10854, 1993.

Acetyl chloride (4.4 mmol, 346 mg) was added to a solution of aluminium chloride (8.5 mmol, 1.13 mg) in dry dichloromethane (10 mL) at 0° C. The reaction mixture was stirred at this temperature for 15 min, cooled to −70° C. and (2-bromo-6-fluorophenyl)-(trimethyl)silane (4.0 mmol, 1.0 g), dissolved in dichloromethane (5 mL), was added. After 4 h at −40° C. the reaction was hydrolyzed with saturated aqueous ammonium chloride, the organic phase separated and the water phase extracted twice with heptane. The combined organic phases were dried over magnesium sulphate, evaporated and purified by silica gel column chromatography (petroleum ether-ethyl acetate) to give the title compound (350 mg).

¹H NMR (400 MHz, CDCl₃): δ 7.41 (1H, d), 7.26 (1H, m), 7.10 (1H, m), 2.60 (3H, s).

GC-MS m/z: 216, 218 [M].

### 4-Bromo-1-(4-fluorophenyl)-3-methyl-1H-indazole

The title compound was obtained from 1-(2-bromo-6-fluorophenyl)ethanone and 4-fluorophenylhydrazine hydrochloride by a method analogous to that described in Example 32 with the following exceptions. The reaction mixture was stirred at 100° C. for 5 days and the final product was further purified by HPLC-C18 to give the title compound.

APCI-MS m/z: 305, 307 [MH+].

### N-((1S)-2-{[1-(4-Fluorophenyl)-3-methyl-1H-indazol-4-yl]amino}-1-methylethyl)-2,4,6-trimethylbenzenesulfonamide

The title compound was obtained from N-[(1S)-2-amino-1-methylethyl]-2,4,6-trimethylbenzenesulfonamide and 4-bromo-1-(4-fluorophenyl)-3-methyl-1H-indazole by a method analogous to that described in Example 2 with the exception that the reaction mixture was stirred for 24 h at 90° C. in an oil bath and the final product was further purified by HPLC-C₁₈ to give the title compound.

¹H NMR (400 MHz, DMSO-d₆): δ 7.72-7.68 (2H, m), 7.63 (1H, bs), 7.42-7.35 (2H, m), 7.50 (1H, t), 6.88-6.83 (3H, m), 5.93 (1H, d) 5.45 (1H, bt), 3.45 (1H, m), 3.19-3.04 (2H, m), 2.63 (3H, s), 2.52 (6H, s), 2.13 (3H, s), 1.02 (3H, s). APCI-MS m/z: 481 [MH⁺].

### EXAMPLE 53

### N-{(1S)-3-[3-(4-Fluorophenyl)-1H-indazol-7-yl]-1-methylpropyl}-2,4,6-trimethylbenzenesulfonamide

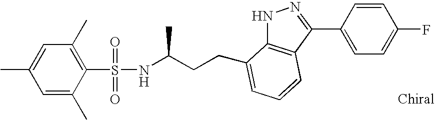

### 3-Bromo-2-fluoro-N-methoxy-N-methyl-benzamide

3-Bromo-2-fluorobenzenecarboxylic acid (4.00 g, 18.0 mmol) was suspended in anhydrous THF (60 mL) and cooled to −30° C. under argon. 4-Methylmorpholine (2.31 mL, 21.0 mmol) was added, followed by dropwise addition of isobutyl chloridocarbonate (2.73 mL, 21.0 mmol). After stirring for 20 min at −30° C., a solution of methoxy(methyl)ammonium chloride (4.11 g, 42.1 mmol) and diisopropylethylamine (7.26 mL, 42.1 mmol) in anhydrous DMF (40 mL) was added. The reaction mixture was allowed to obtain room temperature overnight. After evaporation, the residue was partitioned between water and ethyl acetate. The organic phase was washed with sodium bicarbonate solution and brine. Drying over magnesium sulfate and evaporation gave a residue that was purified by chromatography (SiO2, dichloromethane/ethyl acetate 20/1) to give the title compound as a syrup (3.66 g).

¹H NMR (300 MHz, CDCl₃) δ 7.63 (1H, m), 7.38 (1H, m), 7.09 (1H, m), 3.56 (3H, broad s), 3.36 (3H, broad s); APCI-MS m/z: 261.9, 263.9 [MH].

### (3-Bromo-2-fluoro-phenyl)-(4-fluorophenyl)methanone

p-Fluorophenyl magnesium bromide in diethyl ether (2 M, 6.4 mL, 13 mmol) was added dropwise to a solution of 3-bromo-2-fluoro-N-methoxy-N-methyl-benzamide (2.56 g, 9.76 mmol) in anhydrous THF (40 mL) under argon at −30° C. The reaction mixture was allowed to warm to room temperature overnight. Ethyl acetate (10 mL) was added at −10° C., followed by diethyl ether and 2 M hydrochloric acid to pH 4. The organic phase was dried over magnesium sulfate, evaporated and purified by chromatography (SiO2, light petroleum/dichloromethane 1/1) to give the title compound as a white powder (2.70 g).

¹H NMR (400 MHz, CD₂Cl₂) δ 7.84 (2H, m), 7.76 (1H, m), 7.45 (1H, m), 7.22-7.14 (3H, m).

### 7-Bromo-3-(4-fluorophenyl)-1-H-indazol

A solution of (3-bromo-2-fluoro-phenyl)-(4-fluorophenyl)methanone (500 mg, 1.68 mmol), hydrazine hydrate (0.163 mL, 3.36 mmol) and N,N-dimethyl-4-aminopyridine (41 mg, 0.34 mmol) in pyridine (2 mL) was stirred at 100° C. overnight. Some drops of acetone was added. The reaction mixture was then cooled and partitioned between ethyl acetate and water. The organic phase was washed with sulfuric acid (2M), water and saturated sodium hydrogen carbonate. Drying over magnesium sulfate, evaporation and purification by chromatography (SiO₂, dichloromethane/ethyl acetate 20/1) gave the title compound as a light yellow powder (345 mg).

¹H NMR (400 MHz, CD2Cl₂) δ 7.99-7.93 (3H, m), 7.61 (1H, dd), 7.23 (2H, m), 7.16 (1H, m).

### tert-Butyl (1S,2E)-3-[3-(4-fluorophenyl)-1H-indazol-7-yl]-1-methylprop-2-enylcarbamate

The title compound (102 mg) was prepared from tert-butyl (1S)-1-methylprop-2-enylcarbamate (176 mg, 1.03 mmol) and 7-bromo-3-(4-fluorophenyl)-1-H-indazol (150 mg, 0.515 mmol) by a method analogous to that described in Example 38.

¹H NMR (400 MHz, CD₂Cl₂) δ 7.96 (2H, m), 7.88 (1H, d), 7.40 (1H, d), 7.27-7.18 (3H, m), 6.82 (1H, d, J=16.2 Hz), 6.33 (1H, J₁=5.9 Hz, J₂=16.1 Hz), 4.76 (1H, broad s), 4.44 (1H, m), 1.46 (9H, s), 1.38 (3H, d); APCI-MS m/z: 382.1 [MH⁺].

### tert-Butyl (1S)-3-[3-(4-fluorophenyl)-1H-indazol-7-yl]-1-methylpropylcarbamate

The title compound (96 mg) was prepared from tert-butyl (1S,2E)-3-[3-(4-fluorophenyl)-1H-indazol-7-yl]-1-methylprop-2-enylcarbamate (100 mg, 0.26 mmol) analogously to that described in Example 38.

APCI-MS m/z: 384.1 [MH⁺].

### (1S)-3-[3-(4-Fluorophenyl)-1H-indazol-7-yl]-1-methylpropylamine

The title compound (67 mg) was obtained from tert-butyl (1S)-3-[3-(4-fluorophenyl)-1H-indazol-7-yl]-1-methylpropylcarbamate (94 mg, 0.24 mmol) analogously to that described in Example 39.

APCI-MS m/z: 284.1 [MH⁺].

### N-{(1S)-3-[3-(4-Fluorophenyl)-1H-indazol-7-yl]-1-methylpropyl}-2,4,6-trimethylbenzenesulfonamide

A solution of 2,4,6-benzenesulfonyl chloride (57 mg, 0.26 mmol) in anhydrous THF (1.5 mL) was added dropwise to (1S)-3-[3-(4-fluorophenyl)-1H-indazol-7-yl]-1-methylpropylamine (67 mg, 0.24 mmol) in pyridine (2 mL) at 0° C. The reaction mixture was allowed to reach room temperature overnight, evaporated and purified by HPLC-C₁₈. Conversion into the base form was done by participation between dichloromethane and aqueous sodium hydrogen carbonate. The organic phase was dried over magnesium sulfate. Evaporation and lyophilization from tert-butanol gave the title compound (36 mg).

¹H NMR (400 MHz, CD₂Cl₂) δ 7.95 (2H, m), 7.83 (1H, dd), 7.24-7.11 (4H, m), 6.99 (2H, 2), 4.76 (1H, broad s), 3.40 (1H, m), 2.95 (2H, t), 2.63 (6H, s), 2.31 (3H, s), 1.97 (1H, m), 1.83 (1H, m), 1.10 (3H, d, J=6.6 Hz); APCI-MS m/z: 466.1 [MH⁺].

### EXAMPLE 54

### 2,4,6-Trimethyl-N-[(1S)-1-methyl-3-(1-pyrimidin-5-yl-1H-indazol-4-yl)propyl]benzenesulfonamide

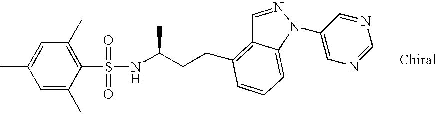

### 4-Bromo-1-(1-pyrimidin-5-yl)-1H-indazole

The title compound was prepared by the method of H.-J. Christau et al., Eur. J. Org. Chem., 2004, 695-709.

A mixture of 5-bromo-1H-indazole (296 mg, 1.5 mmol), 5-bromopyrimidine (477 mg, 3.0 mmol), salicylaldoxime (41 mg, 0.3 mmol), copper(I) oxide (11 mg, 0.075 mmol) and cesium carbonate (1.47 g, 4.5 mmol) in acetonitrile (6 mL) was stirred under argon at 82° C. overnight. The mixture was diluted with dichloromethane, filtered through celite, concentrated and purified by chromatography (SiO₂, dichloromethane/ethyl acetate 5/1) to give the title compound as a white powder (100 mg).

¹H NMR (400 MHz, CD₂Cl₂) δ 9.20 (3H, m), 8.33 (1H, s), 7.74 (1H, m), 7.49 (1H, m), 7.41 (1H, m).

### tert-Butyl (1S,2E)-3-(1-pyrimidin-5-yl-1H-indazol-4-yl)-1-methylprop-2-enylcarbamate

The title compound (46 mg) was prepared from tert-butyl (1S)-1-methylprop-2-enylcarbamate (121 mg, 0.705 mmol) and 4-bromo-1-(1-pyrimidin-5-yl)-1H-indazole (97 mg, 0.35 mmol) by a method analogous to that described in Example 38.

¹H NMR (400 MHz, CD₂Cl₂) δ 9.24 (2H, broad s), 9.17 (1H, broad s), 8.47 (1H, m), 7.67 (1H, m), 7.50 (1H, m), 7.36 (1H, m), 6.90 (1H, m), 6.47 (1H, dd, J₁=5.6 Hz, J₂=15.9 Hz), 4.70 (1H, broad s), 4.47 (1H, broad s), 1.46 (9H, s), 1.38 (3H, d, J=6.9 Hz).

### tert-Butyl (1S)-3-(1-pyrimidin-5-yl-1H-indazol-4-yl)-1-methylpropylcarbamate

Preparation from tert-butyl (1S,2E)-3-(1-pyrimidin-5-yl-1H-indazol-4-yl)-1-methylprop-2-enylcarbamate (45 mg, 0.12 mmol), analogously to that described in Example 38, followed by purification by HPLC-C₁₈ gave the title compound (41 mg).

APCI-MS m/z: 368.1 [MH⁺].

### (1S)-3-(1-Pyrimidin-5-yl-1H-indazol-4-yl)-1-methylpropylamine

The title compound (19 mg) was obtained from tert-butyl (1S)-3-(1-pyrimidin-5-yl-1H-indazol-4-yl)-1-methylpropylcarbamate (41 mg, 0.11 mmol) analogously to that described in Example 39.

APCI-MS m/z: 268.1 [MH⁺].

### 2,4,6-Trimethyl-N-[(1S)-1-methyl-3-(1-pyrimidin-5-yl-1H-indazol-4-yl)propyl]benzenesulfonamide

(1S)-3-(1-Pyrimidin-5-yl-1H-indazol-4-yl)-1-methylpropylamine (19 mg, 0.071 mmol) was reacted with 2,4,6-benzenesulfonyl chloride (39 mg, 0.18 mmol) by a method analogous to that described in Example 38. Purification by HPLC-C₁₈, followed by lyophilisation from dioxane gave the title compound (26 mg).

¹H NMR (400 MHz, CD₂Cl₂) δ 9.24 (2H, broad s), 9.16 (1H, broad s), 8.18 (1H, s), 7.62 (1H, d, J=8.4 Hz), 7.41 (1H, dd, J₁=7.2 Hz, J₂=8.4 Hz), 6.99-6.92 (3H, m), 4.48 (1H, broad d), 3.35 (1H, m), 3.02-2.81 (2H, m), 2.60 (6H, s), 2.29 (3H, s), 1.90-1.75 (2H, m), 1.13 (1H, d, J=6.7 Hz); APCI-MS m/z: 450.1 [MH].

### EXAMPLE 55

### N-[2-[[1-(4-Fluorophenyl)indazol-4-yl]amino]-2-methylpropyl]-2,4,6-trimethyl-benzenesulfonamide

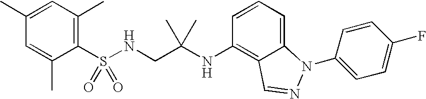

### 2,2-Dimethylaziridin-1-yl-phenylmethanone

The title compound was prepared using the method of C. W. Woods et al., J. Med. Chem. 7, 371-373, 1964):

2,2-Dimethylaziridine (1.78 g, 25 mmol; prepared according to T. L. Cairns, J. Am. Chem. Soc. 63, 870-871, 1941) was dissolved in dichloromethane (25 mL). Aqueous sodium hydroxide (4M, 6.25 mL) was added and the mixture was stirred at −10° C. Benzoyl chloride (3.52 g, 25 mmol) was added during 5 min and stirring was continued for 1 min at −10° C. The temperature was then allowed to rise to 5° C. during 70 min. The phases were separated and the organic phase was washed with water, saturated aqueous sodium chloride and evaporated to give the title compound as a colourless liquid (3.83 g, 87%) sufficiently pure for the next synthetic transformation.

¹H-NMR (300 MHz, CDCl₃): 8.00-7.95 (2H, m), 7.55 (1H, tt), 7.50-7.42 (2H, m), 2.34 (2H, s), 1.28 (6H, s)

### 1-(4-Fluorophenyl)-4-nitroindazole

2,6-Dinitrobenzaldehyde (2.6 g, 13.3 mmol) and (4-fluorophenyl)hydrazine hydrochloride (2.2 g, 13.5 mmol) was dissolved in DMF (30 mL). Cesium carbonate (12.2 g, 37.4 mmol) was added and the mixture was vigorously stirred for 1 h. Water was then added and the precipitate filtered off, washed with water and dried in vacuum to give the title compound as yellow needles (3.03 g, 88%). An analytical sample was re-crystallised from ethanol.

m.p. 199-200° C.

¹H-NMR (300 MHz, DMSO-d₆): 8.80 (1H, d), 8.26 (2H, dd), 7.88-7.80 (2H, m), 7.73 (1H, t), 7.53-7.54 (2H, m).

¹⁹F-NMR (DMSO-d₆): −113.81 (tt)

### 1-(4-Fluorophenyl)indazol-4-amine

The title compound was prepared using the method described by Broggini et al, Tet. Asymmetry. 10, 2203-2212, 1999:

1-(4-Fluorophenyl)-4-nitroindazole (3.12 g, 12.1 mmol) was dissolved in ethanol (40 mL). Iron powder (5.4 g, 96 mmol) and aqueous acetic acid (20%, 6 mL) was added. The mixture was stirred at reflux for 35 min and then cooled, diluted with ethyl acetate and filtered through celite. The filtrate was washed with saturated aqueous sodium hydrogen carbonate, water and finally dried over sodium sulphate. Filtering, evaporation and crystallization from methanol-water gave the title compound as beige needles (monohydrate, 2.28 g, 76%).

m.p. 84-88° C.

¹H-NMR (300 MHz, DMSO-d₆): 8.39 (1H, d), 7.78-7.70 (2H, m), 7.43-7.34 (2H, m), 7.13 (1H, dd), 6.85 (1H, d, further coupled), 6.28 (1H, d, further coupled), 5.99 (2H, s, NH₂)

¹⁹F-NMR (DMSO-d₆): −116.41 (tt)

APCI-MS m/z: 228.0 [MH⁺].

A sample (978.3 mg, 4 mmol assuming a monohydrate) was dried in vacuo at 40° C. to constant weight (898.5 mg). The weight loss corresponds to the loss of 4.4 mmol of water. During the process the beige, crystalline material transformed into a light brown powder.

### N-[2-[[1-(4-Fluorophenyl)indazol-4-yl]amino]-2-methylpropyl]benzamide

1-(4-Fluorophenyl)indazol-4-amine (anhydrous, 670 mg, 2.95 mmol) was dissolved in methanol (5 mL) and 2,2-dimethylaziridin-1-yl-phenylmethanone (1.5 g, 8.6 mmol) was added. The mixture was stirred at ambient temperature for 8 d after which time additional 2,2-dimethylaziridin-1-yl-phenylmethanone (0.4 g, 2.3 mmol) was added. The S_{N}1 type reaction proceeded very slowly (c.f. Lin et al, Tetrahedron 48 (12), 2359-2372, 1992) to yield the title compound and a side product, N-(2-methoxy-2-methyl-propyl)benzamide, in approximately equal amounts. After stirring for a total of 13 d, the mixture was pooled with a similar batch prepared from 1-(4-fluorophenyl)indazol-4-amine (253 mg) and 2,2-dimethylaziridin-1-yl-phenylmethanone (611 mg) in methanol (0.5 mL). The pooled reaction mixtures were evaporated and subjected to flash chromatography (SiO₂, 1080% ethyl acetate in heptane) to give a material consisting of the title compound, methyl ether side product and the starting indazole. Separation was performed using preparative HPLC (C-18, gradient CH₃CN—H₂O, 0.1% TFA). Fractions containing the title compound were evaporated free of CH₃CN and the aqueous residue was then made basic with an excess of aqueous sodium hydroxide (2M) and extracted with ethyl acetate. The organic phase was washed with water, saturated aqueous sodium chloride and then evaporated to give the pure title compound (538 mg, 32%).

¹H-NMR (300 MHz, DMSO-d₆): 8.88 (1H, t, amide NH), 8.40 (1H, s), 7.95-7.90 (2H, m), 7.75-7.69 (2H, m), 7.59-7.53 (1H, m), 7.53-7.47 (2H, m), 7.39 (2H, t, further coupled), 7.20 (1H, t), 6.99 (1H, d), 6.48 (1H, d), 6.38 (1H, s, NH), 3.58 (2H, d), 1.46 (6H, s).

APCI-MS m/z 403.1 [MH⁺].

### N-[1-(4-Fluorophenyl)indazol-4-yl]-2-methylpropane-1,2-diamine

N-[2-[[1-(4-Fluorophenyl)indazol-4-yl]amino]-2-methylpropyl]benzamide (330 mg, 1.1 mmol) was suspended in hydrochloric acid (4 M, 110 mL) and refluxed for 5.5 h. After cooling, the clear solution was washed twice with dichloromethane and an excess of aqueous sodium hydroxide (10 M) was added. The basic aqueous suspension was then extracted with ethyl acetate and dichloromethane and the pooled organic phases were washed with water, saturated aqueous sodium chloride and evaporated. The residue was dissolved in dichloromethane, filtered free of residual sodium chloride and evaporated to give the title compound (223 mg, 91%) as an oil that crystallised as low melting needles when stored at 4° C.

¹H-NMR (400 MHz, DMSO-d₆): 8.52 (1H, s), 7.77-7.69 (2H, m), 7.39, (2H, t, further coupled), 7.18 (1H, t), 6.89 (1H, d), 6.45 (1H, d), 5.63 (1H, s, NH), 2.74 (2H, s), 1.60 (2H, bs, NH₂), 1.34 (6H, s)

### N-[2-[[1-(4-fluorophenyl)indazol-4-yl]amino]-2-methylpropyl]-2,4,6-trimethyl-benzenesulfonamide

N-[1-(4-Fluorophenyl)indazol-4-yl]-2-methylpropane-1,2-diamine (45.3 mg, 0.15 mmol) was dissolved in pyridine (6 mL) and the solution was cooled to 0° C. (c.f. Sulkowski & Mascitti U.S. Pat. No. 3,931,218). A solution of 2,4,6-trimethyl-benzenesulfonylchloride (36 mg, 0.16 mmol) was added in portions during 0.5 min. The mixture was stirred at 0° C. for 25 min. the cooling was then removed and additional 2,4,6-trimethyl-benzenesulfonylchloride (15 mg, 0.07 mmol) was added. After stirring for 75 min at ambient temperature the reaction was quenched by adding saturated aqueous ammonium chloride (6 drops) and the mixture was co-evaporated with toluene. The residue was subjected to flash chromatography (SiO₂, 10→90% ethyl acetate in heptane) and fractions containing the title compound were further purified by preparative HPLC (C-18, CH₃CN—H₂O, 0.1% TFA). After evaporation of acetonitrile, saturated aqueous sodium hydrogen carbonate was added and the mixture was extracted twice with ethyl acetate. Evaporation and crystallization from ethyl acetate-heptane gave the title compound as needles (26 mg, 35%).

m.p. 155.5-156.5

¹H-NMR (400 MHz, DMSO-d₆): 8.43 (1H, d), 7.76-7.70 (2H, d), 7.62 (1H, bs, NH), 7.40 (2H, t, further coupled), 7.09 (1H, t), 6.95 (2H, s), 6.89 (1H, d), 6.26 (1H, d), 5.61 (1H, s, NH), 3.02 (2H, s), 2.54 (6H, s), 2.20 (3H, s), 1.32 (6H, s).

¹⁹F-NMR (DMSO-d₆): −116.16 (tt)

APCI-MS m/z 481.1 [MH⁺].

### EXAMPLE 56

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10× (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10× (Panvera, Part number P2814, stored at −70° C. initially but once thawed stored at room temperature). Avoid repeated freeze/thaws for all reagents. The GR Screening buffer 10× comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (8 μL; assay buffer 10×, Stabilizing Peptide, DTT and ice cold MQ water) was added to the background wells. GS Red solution (7 μL; assay buffer 10×, Stabilizing Peptide, DTT, GS Red and ice cold water) was added to all wells except background wells. GR solution (7 μL; assay buffer 10×, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nM and a dichroic mirror at 561 nm). The IC50 values were calculated using XLfit model 205.

| | GRhuFL_FP_v2 (GR-binders) |
|---|---|
| Example No | IC50 (nM) |
| 1 | 2.9 |
| 2 | 2.9 |
| 3 | 2.3 |
| 4 | 4.0 |
| 5 | 5.4 |
| 6 | 15 |
| 7 | 3.5 |
| 8 | 6.9 |
| 9 | 3.8 |
| 10 | 7.1 |
| 11 | 6.6 |
| 12 | 3.9 |
| 13 | 4.0 |
| 14 | 4.3 |
| 15 | 5.4 |
| 16 | 5.6 |
| 17 | 4.0 |
| 18 | 57 |
| 19 | 260 |
| 20 | 4.4 |
| 21 | 2.7 |
| 22 | 3.5 |
| 23 | 7.3 |
| 24 | 8.7 |
| 25 | 3.8 |
| 26 | 3.0 |
| 27 | 12 |
| 28 | 23 |
| 29 | 5.7 |
| 30 | 4.7 |
| 31 | 44 |
| 32 | 4.2 |
| 33 | 5.5 |
| 34 | 5300 |
| 35 | 8.0 |
| 36 | 7.6 |
| 37 | 790 |
| 38 | 45 |
| 39 | 4.6 |
| 40 | 6.4 |
| 41 | 1.50 |
| 42 | 4000 |
| 43 | 13 |
| 44 | 80 |
| 45 | 18 |
| 46 | 18 |
| 47 | 36 |
| 48 | 51 |
| 49 | 54 |
| 50 | 7.0 |
| 51 | 20 |
| 52 | 272 |
| 53 | 10 |
| 54 | 17 |
| 55 | 24 |

1. A compound of formula (I): 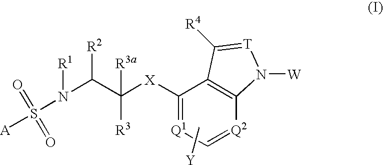
wherein:
A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ haloalkyl, C₁₋₄ haloalkoxy, C₃₋₆ cycloalkyl, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹), pyrazolyl (optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹) or tetrahydrofuranyl (optionally substituted by C₁₋₆ alkyl);
R¹⁰, R¹¹, R¹⁴, R¹⁵, R¹⁶, R¹⁷, R¹⁸, R¹⁹, R²⁰ and R²¹ are, independently, hydrogen, C₁₋₄ alkyl or C₃₋₇ cycloalkyl;
R¹ is hydrogen;
R² is hydrogen, C₁₋₄ alkyl or C₁₋₄ haloalkyl, C₃₋₇ cycloalkyl or C₃₋₇ cyclohaloalkyl;
R³ is hydrogen, C₁₋₄ alkyl or C₁₋₄ haloalkyl;
R^{3a} is hydrogen or C₁₋₄ alkyl;
R⁴ is hydrogen, halogen, C₁₋₄ alkyl or C₁₋₄ haloalkyl;
T is CH or N;
Q¹ is CY¹ or N;
Q² is CY² or N;
W is phenyl, C₃₋₇ cycloalkyl, thienyl, isoxazolyl, pyrazolyl, pyridinyl or pyrimidinyl all of which are optionally substituted by halo, C₁₋₆ alkyl (optionally substituted by C₁₋₆ alkoxy), C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³;
X is CH₂, O, S, S(O), S(O)₂ or NH;
Y, Y¹ and Y² are, independently, hydrogen, halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR²²R²³;
R¹², R¹³, R²² and R²³ are, independently, hydrogen, C₁₋₄ alkyl or C₃₋₇ cycloalkyl;
or a pharmaceutically acceptable salt thereof.

2. A compound of formula (I) as claimed in claim 1 wherein R¹ is hydrogen.

3. A compound of formula (I) as claimed in claim 1 wherein R² is methyl, ethyl or C₁₋₂ fluoroalkyl.

4. A compound of formula (I) as claimed in claim 1 wherein R³ is hydrogen.

5. A compound of formula (I) as claimed in claim 1 wherein R^{3a} is hydrogen.

6. A compound of formula (J) as claimed in claim 1 wherein R⁴ is hydrogen.

7. A compound of formula (I) as claimed in claim 1 wherein T is N.

8. A compound of formula (I) as claimed in claim 1 wherein Q¹ is CY¹ and Q² is CY².

9. A compound of formula (I) as claimed in claim 1 wherein Y is hydrogen.

10. A compound of formula (I) as claimed in claim 1 wherein Y¹ and Y² are both hydrogen.

11. A compound of formula (I) as claimed in claim 1 wherein A is phenyl (optionally substituted by halogen, C₁₋₄ alkyl, C₁₋₄ haloalkyl, C₁₋₄ alkoxy or C₁₋₄ haloalkoxy).

12. A compound of formula (I) as claimed in claim 1 wherein W is phenyl, pyridinyl or pyrimidinyl all of which are optionally substituted by halogen, C₁₋₄ alkyl (optionally substituted by C₁₋₄ alkoxy), C₁₋₄ alkoxy, C₁₋₄ fluoroalkyl, C₁₋₄ fluoroalkoxy, CN or CO₂H.

13. A compound:
N-((1S)-2-{[1-(4-Fluorophenyl)-1H-indazol-4-yl]oxy}-1-methylethyl)-2,4,6-trimethylbenzenesulfonamide;
N-[(1S)-2-[[1-(4-Fluorophenyl)-1H-indazol-4-yl]amino]-1-methylethyl]-2,4,6-trimethyl-benzenesulfonamide;
N-((1S)-2-{[1-(6-Fluoropyridin-3-yl)-1H-indazol-4-yl]amino}-1-methylethyl)-2,4,6-trimethylbenzenesulfonamide;
2,4,6-Trimethyl-N-[2,2,2-trifluoro-1-({[1-(6-fluorophenyl)-1H-indazol-4-yl]oxy}methyl)ethyl]benzenesulfonamide;
N-((1S)-2-{[1-(4-Methoxyphenyl)-1H-indazol-4-yl]amino}-1-methylethyl)-2,4,6-trimethylbenzenesulfonamide;
2,4,6-Trimethyl-N-[(1S)-1-methyl-2-({1-[3-(trifluoromethoxy)phenyl]-1H-indazol-4-yl}amino)ethyl]benzenesulfonamide;
2,4,6-Trimethyl-N-{(1S)-1-methyl-2-[(1-phenyl-1H-indazol-4-yl)amino]ethyl}benzenesulfonamide;
N-((1S)-2-{[1-(3-Methoxyphenyl)-1H-indazol-4-yl]amino}-1-methylethyl)-2,4,6-trimethylbenzenesulfonamide;
2,4,6-Trimethyl-N-((1S)-1-methyl-2-{[1-(3-methylphenyl)-1H-indazol-4-yl]amino}ethyl)benzenesulfonamide;
N-((1S)-2-{[1-(2-Fluoropyridin-4-yl)-1H-indazol-4-yl]amino}-1-methylethyl)-2,4,6-trimethylbenzenesulfonamide;
N-((1S)-2-{[1-(6-Methoxypyridin-3-yl)-1H-indazol-4-yl]amino}-1-methylethyl)-2,4,6-trimethylbenzenesulfonamide;
2,4,6-Trimethyl-N-((1S)-1-methyl-2-{[1-(4-methylphenyl)-1H-indazol-4-yl]amino}ethyl)benzenesulfonamide;
N-((1S)-2-{[1-(3-Fluorophenyl)-1H-indazol-4-yl]amino}-1-methylethyl)-2,4,6-trimethylbenzenesulfonamide;
2,4,6-Trimethyl-N-{(1S)-1-methyl-2-[(1-pyridin-4-yl-1H-indazol-4-yl)amino]ethyl}benzenesulfonamide;
2,4,6-Trimethyl-N-{(1S)-1-methyl-2-[(1-pyrimidin-5-yl-1H-indazol-4-yl)amino]ethyl}benzenesulfonamide;
2,4,6-Trimethyl-N-{(1S)-1-methyl-2-[(1-pyridin-3-yl-1H-indazol-4-yl)amino]ethyl}benzenesulfonamide;
N-((1S)-2-{[1-(4-Fluoro-3-methylphenyl)-1H-indazol-4-yl]amino}-1-methylethyl)-2,4,6-trimethylbenzenesulfonamide;
3-[4-({(2S)-2-[(2,4,6-benzenesulfonyl)amino]propyl}amino)-1H-indazol-1-yl]benzoic acid;
2,4,6-Trimethyl-N-[(1S)-1-methyl-2-({1-[3-(trifluoromethyl)phenyl]-1H-indazol-4-yl}amino)ethyl]benzenesulfonamide;
N-[(1S)-2-({1-[3-(Methoxymethyl)phenyl]-1H-indazol-4-yl}amino)-1-methylethyl]-2,4,6-trimethylbenzenesulfonamide;
N-((1S)-2-{[1-(3-Fluoro-4-methoxyphenyl)-1H-indazol-4-yl]amino}-1-methylethyl)-2,4,6-trimethylbenzenesulfonamide;
N-((1S)-2-{[1-(4-Chlorophenyl)-1H-indazol-4-yl]amino}-1-methylethyl)-2,4,6-trimethylbenzenesulfonamide;
N-((1S)-2-{[1-(4-Fluorophenyl)-5-methyl-1H-indazol-4-yl]amino}-1-methylethyl)-2,4,6-trimethylbenzenesulfonamide;
N-((2R)-2-{[1-(4-Fluorophenyl)-1H-indazol-4-yl]amino}propyl)-2,4,6-trimethylbenzenesulfonamide;
1-Cyclopentyl-N-((1S)-2-{[1-(4-fluorophenyl)-1H-indazol-4-yl]amino}-1-methylethyl)-3,5-dimethyl-1H-pyrazole-4-sulfonamide;
1-Cyclopentyl-N-((1S)-2-{[1-(6-fluoropyridin-3-yl)-1H-indazol-4-yl]amino}-1-methylethyl)-3,5-dimethyl-1H-pyrazole-4-sulfonamide;
1-Cyclopentyl-3,5-dimethyl-N-[(1S)-1-methyl-2-({1-[4-(trifluoromethoxy)phenyl]-1H-indazol-4-yl}amino)ethyl]-1H-pyrazole-4-sulfonamide;
1-Cyclopentyl-N-((1S)-2-{[1-(2-methoxypyrimidin-5-yl)-1H-indazol-4-yl]amino}-1-methylethyl)-3,5-dimethyl-1H-pyrazole-4-sulfonamide;
1-Cyclopentyl-3,5-dimethyl-N-{(1S)-1-methyl-2-[(1-pyrimidin-5-yl-1H-indazol-4-yl)amino]ethyl}-1H-pyrazole-4-sulfonamide;
N-((1S)-2-{[1-(4-Cyanophenyl)-1H-indazol-4-yl]amino}-1-methylethyl)-1-cyclopentyl-3,5-dimethyl-1H-pyrazole-4-sulfonamide;
1-Cyclopentyl-N-((1S)-2-{[1-(5-methoxypyridin-3-yl)-1H-indazol-4-yl]amino}-1-methylethyl)-3,5-dimethyl-1H-pyrazole-4-sulfonamide;
N-((1S)-2-{[5-Fluoro-1-(4-fluorophenyl)-1H-indazol-4-yl]amino}-1-methylethyl)-2,4,6-trimethylbenzenesulfonamide;
N-((1S)-2-{[7-Fluoro-1-(4-fluorophenyl)-1H-indazol-4-yl]amino}-1-methylethyl)-2,4,6-trimethylbenzenesulfonamide;
2,4,6-Trimethyl-N-{(1S)-1-methyl-2-[(1-phenyl-1H-pyrazolo[3,4-d]pyrimidin-4-yl)amino]ethyl}benzenesulfonamide;
N-[(1S)-1-({[1-(4-Fluorophenyl)-1H-indazol-4-yl]amino}methyl)-2-methylpropyl]-2,4,6-trimethylbenzenesulfonamide;
N-[2-[1-(4-Fluorophenyl)indazol-4-yl]sulfanyl-1-methylethyl]-2,4,6-trimethyl-benzenesulfonamide;
N-[2-[1-(4-Fluorophenyl)indazol-4-yl]sulfonyl-1-methylethyl]-2,4,6-trimethyl-benzenesulfonamide;
N-{3-[1-(4-Fluorophenyl)-1H-indazol-4-yl]-1-methylpropyl}-2,4,6-trimethylbenzenesulfonamide;
N-{(1S)-3-[1-(4-Fluorophenyl)-1H-indazol-4-yl]-1-methylpropyl}-2,4,6-trimethylbenzenesulfonamide;
N-((2S)-2-{[1-(4-Fluorophenyl)-1H-indazol-4-yl]amino}propyl)-2,4,6-trimethylbenzenesulfonamide;
N-((1S)-2-{[1-(4-Fluorophenyl)-1H-indazol-4-yl]oxy}-1-methylethyl)-3,5-dimethyl-1H-pyrazole-4-sulfonamide;
3,5-Dimethyl-N-{(1S)-1-methyl-2-[(1-pyridin-3-yl-1H-indazol-4-yl)oxy]ethyl}-1H-pyrazole-4-sulfonamide;
1-tert-Butyl-N-((1S)-2-{[1-(4-fluorophenyl)-1H-indazol-4-yl]oxy}-1-methylethyl)-3,5-dimethyl-1H-pyrazole-4-sulfonamide;
1-tert-Butyl-3,5-dimethyl-N-{(1S)-1-methyl-2-[(1-pyridin-3-yl-1H-indazol-4-yl)oxy]ethyl}-1H-pyrazole-4-sulfonamide;
N-((1S)-2-{[1-(4-Fluorophenyl)-1H-indazol-4-yl]oxy}-1-methylethyl)-3,5-dimethyl-1-[(3R)-tetrahydrofuran-3-yl]-1H-pyrazole-4-sulfonamide;
1-(1-Ethylpropyl)-N-((1S)-2-{[1-(4-fluorophenyl)-1H-indazol-4-yl]oxy}-1-methylethyl)-3,5-dimethyl-1H-pyrazole-4-sulfonamide;
N-((1S)-2-{[1-(4-Fluorophenyl)-1H-indazol-4-yl]oxy}-1-methylethyl)-3,5-dimethyl-1-[(3S)-tetrahydrofuran-3-yl]-1H-pyrazole-4-sulfonamide;
1-Cyclopentyl-N-((1S)-2-{[1-(4-fluorophenyl)-1H-indazol-4-yl]oxy}-1-methylethyl)-3,5-dimethyl-1H-pyrazole-4-sulfonamide;
1-Cyclopentyl-3,5-dimethyl-N-{(1S)-1-methyl-2-[(1-pyridin-3-yl-1H-indazol-4-yl)oxy]ethyl}-1H-pyrazole-4-sulfonamide;
N-{(1S)-2-[(1-Cyclopentyl-1H-indazol-4-yl)amino]-1-methylethyl}-2,4,6-trimethylbenzenesulfonamide;
N-((1S)-1-ethyl-2-{[1-(4-methylphenyl)-1H-indazol-4-yl]amino}ethyl)benzenesulfonamide;
N-((1S)-2-{[1-(4-Fluorophenyl)-3-methyl-1H-indazol-4-yl]amino}-1-methylethyl)-2,4,6-trimethylbenzenesulfonamide;
N-{(1S)-3-[3-(4-Fluorophenyl)-1H-indazol-7-yl]-1-methylpropyl}-2,4,6-trimethylbenzenesulfonamide;
2,4,6-Trimethyl-N-[(1S)-1-methyl-3-(1-pyrimidin-5-yl-1H-indazol-4-yl)propyl]benzenesulfonamide; or,
N-[2-[[1-(4-Fluorophenyl)indazol-4-yl]amino]-2-methylpropyl]-2,4,6-trimethyl-benzenesulfonamide;
or a pharmaceutically acceptable salt thereof.

14. A process for the preparation of a compound of formula (I) as claimed in claim 1, the process comprising:
a. coupling a compound of formula (II): 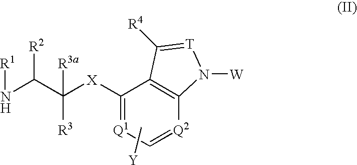
with a compound of formula (III): 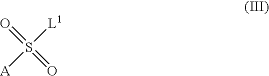
wherein L¹ is a leaving group, in a suitable solvent, in the presence of a suitable base and at a suitable temperature;
b. coupling a compound of formula (IV): 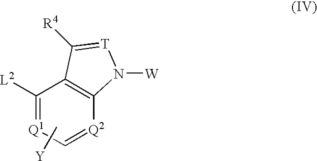
wherein L² is a leaving group, with a compound of formula (V): 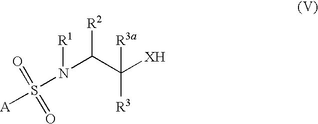
in a suitable solvent, in the presence of a suitable base and at a suitable temperature; or,
c. coupling a compound of formula (VI): 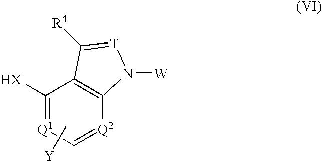
with a compound of formula (VII): 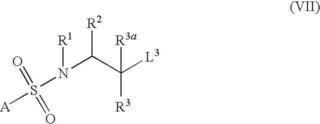
wherein L³ is a leaving group, in a suitable solvent, in the presence of a suitable base and at a suitable temperature.

15. A pharmaceutical composition comprising a compound or formula (I) or a pharmaceutically acceptable salt thereof as defined in claim 1, and a pharmaceutically acceptable adjuvant, diluent or carrier.

16. (canceled)

17. (canceled)

18. A method of treating a glucocorticoid receptor mediated disease state in a mammal, which comprises administering to a mammal in need of such treatment an effective amount of a compound of formula (I), or a pharmaceutically acceptable salt thereof as claimed in claim 1.

19. A combination of a compound of formula (I), or a pharmaceutically acceptable salt thereof as claimed in claim 1, and one or more agents selected from the list comprising:
a PDE4 inhibitor including an inhibitor of the isoform PDE4D;
a selective β.sub2. adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
a muscarinic receptor antagonist (for example a M1, M2 or M3 antagonist, such as a selective M3 antagonist) such as ipratropium bromide, tiotropium bromide, oxitropium bromide, pirenzepine or telenzepine;
a modulator of chemokine receptor function (such as a CCR1 receptor antagonist); or,
an inhibitor of p38 kinase function.
